(19) United States
(12) Patent Application Publication
(10) Pub. No.: US 2025/0222279 A1
(43) Pub. Date: Jul. 10, 2025

(54) ROTATABLE SUBJECT SUPPORT APPARATUS

(71) Applicant: Elekta Beijing Medical Systems Ca., Ltd., Beijing (CN)

(72) Inventors: Kjell Eldered, Beijing (CN); Xun FENG, Beijing (CN); Junlin FU, Beijing (CN); Changhong LIU, Beijing (CN); Zhen XIN, Beijing (CN)

(21) Appl. No.: 18/850,994

(22) PCT Filed: Mar. 25, 2022
(86) PCT No.: PCT/CN2022/083020

§ 371 Date: Sep. 25, 2024

(51) Int. Cl. A61N 5/10

(52) U.S. Cl. CPC A61N 5/1082

(57) ABSTRACT

A subject support apparatus (1400, 1600) for a radiation therapy device. A subject support apparatus (1400) comprises a subject support (1410), a subject support receiving portion (1420) and a deformable guide (1418). The subject support (1410) is configured to tilt relative to the subject support receiving portion (1420). The deformable guide (1418) comprises a first face (1431) and a second face (1433), wherein, throughout a range of tilt configurations, the first face (1431) is arranged to be in contact, at least in part, with a longitudinal side wall of the subject support (1410). A subject support apparatus (1600) comprises a platform (1618), a subject support movable relative to the platform (1618) and an interface board (1646) movable relative to the platform (1618), wherein the interface board (1646) is physically coupled to the subject support such that a translation of the subject support relative to the platform (1618) causes a translation of the interface board (1646), and wherein the interface board (1646) is configured to electrically connect the subject support to an electrical cable. A device for a subject support apparatus (114) comprises a linkage (220) for moving a platform (214), a base (228) movably coupled to the linkage (220), wherein the base (228) comprises a linkage receiving region configured to receive at least a portion of the linkage (220) when the linkage (220) is in the retracted pose, and wherein said portion of the linkage (220) extends beyond the base (228) when the linkage (220) is in the extended pose.

The present disclosure relates generally to a radiotherapy apparatus, and in particular to a subject support for a radiotherapy apparatus for positioning a subject during the delivery or application of radiotherapy.

### BACKGROUND

Radiotherapy and medical imaging are becoming increasingly common when treating many types of cancers. As such, increasing numbers of patients are undergoing treatment or imaging, and require the use of radiotherapy apparatuses. Many patients of these apparatuses have health issues, resulting in weakness, reduced mobility, and the like. Radiotherapy treatments are also highly dependent on delivering a dose of radiation accurately, and the positioning of the patient is therefore important to ensure that tumours are targeted and healthy tissue is not destroyed.

Although much research has been done to mitigate the risk to healthy tissue associated with radiotherapy, the correct positioning of a patient during treatment and scanning is key to ensuring that the prescribed dose of radiation is delivered accurately and optimally to the target region. Components of a radiotherapy apparatus are therefore configured to move the patient accordingly. A subject support apparatus may be used to position a patient whilst the patient is being scanned by a medical device, e.g. a medical imaging device, or whilst the patient is receiving treatment, e.g. radiotherapy. The subject support apparatus may be a bed or couch on which the patient is positioned. The subject support apparatus may be moved through multiple degrees of freedom, allowing the patient to be appropriately positioned prior to and during treatment or imaging.

In order to enable the positioning of the patient, existing systems typically use a large number of moving parts which are connected together. However, the use of so many moving parts introduces further risks to both the patient and the equipment. For example, limbs such as fingers or arms of a patient may be pinched or trapped when the subject support apparatus tilts or rotates. Cables, tubes and other components may similarly be pinched, broken or otherwise compromised during movement or rotation of various components of the subject support apparatus.

There is therefore a need to improve the accessibility of subject support apparatuses for patients, particularly considering patient weakness, reduced mobility, reduced health and the like, as well as the growing range of patients being treated using these treatments, and to improve the overall safety and maintenance of the subject support apparatus and additional radiotherapy devices.

The present invention(s) seek to address these and other disadvantages.

### SUMMARY

Aspects of the invention are defined in the independent claims. Optional features are defined in the dependent claims.

According to an aspect, a device for a subject support apparatus is provided, wherein the device comprises a linkage for moving a platform, the linkage being movable between an extended pose and a retracted pose, and a base movably coupled to the linkage, wherein the base comprises a linkage receiving region configured to receive at least a portion of the linkage when the linkage is in the retracted pose, and wherein said portion of the linkage extends beyond the base when the linkage is in the extended pose.

The linkage receiving region may comprise a recess.

The linkage receiving region may comprise a cut-out portion of the base.

The linkage receiving region can be defined by having a thickness less than a total thickness of the base, measured vertically.

The base can be movably coupled to the linkage via a coupling disposed in the linkage receiving region.

The linkage may comprise a first member movably coupled to the base, and the base may be configured to receive at least a portion of the first member. The first member may have a length and a width, and the received portion of the first member may extend along the length of the first member.

The linkage can further comprise a second member movably coupled to the first member, wherein the second member is movably couplable to a platform.

The linkage can be configured to adjust at least one of the height, pitch, roll and yaw of a subject support platform.

The linkage may be disposed under a platform of the subject support apparatus.

The linkage and base can be configured in a stacked arrangement with a platform of the subject support apparatus.

The base can be configured to be movably couplable to a floor of a treatment room.

The linkage may be configured to raise and/or lower a platform.

The recess may extend towards a bottom surface of the base.

A subject support apparatus is provided according to any of the above implementations. The subject support apparatus may further comprise a platform.

According to a second aspect, a subject support apparatus for a radiation therapy device is provided, wherein the apparatus comprises a subject support receiving portion comprising a longitudinal interior wall and a longitudinal exterior wall, the longitudinal exterior wall being inclined relative to the horizontal axis. The apparatus further comprises a subject support comprising a longitudinal side wall, and a tabletop component comprising a surface and an extending edge, the extending edge extending beyond the longitudinal side wall, the subject support being tiltable about a tilt axis relative to the subject support receiving portion, wherein the longitudinal side wall is arranged to oppose the longitudinal interior wall of the subject support receiving portion, and wherein, at a maximally tilted position of the subject support, a distance between a lower surface of the extending edge and the longitudinal exterior wall increases with increasing displacement from a central longitudinal axis of the subject support.

The longitudinal exterior wall can be inclined at a first angle relative to the horizontal axis and, at the maximally tilted position of the subject support, the extending edge is angled at a second angle relative to the horizontal axis, and a difference between the first angle and the second angle is greater than a threshold angular difference.

The extending edge can be in-plane with the surface of the tabletop component.

The subject support receiving portion may comprise a cover.

The extending edge of the tabletop component can further comprise a recess for receiving at least part of an accessory attached to the subject support.

The recess can comprise an accessory surface located on a side of the subject support which is opposite to the surface of the tabletop component.

The longitudinal side wall can comprise a portion which does not oppose the longitudinal interior wall of the subject support receiving portion as the subject support is tilted about the axis.

The extending edge of the tabletop component of the subject support may comprise a liquid directing feature for minimising liquid ingress into the subject support receiving portion.

The liquid directing feature may comprise a groove configured to interrupt flow of liquid from the subject support towards the subject support receiving portion.

The groove may be located on the underside of the extending edge, and may be arranged to provide capillary action.

The liquid directing feature may run substantially parallel to the tilt axis.

The liquid directing feature can be configured to direct liquid away from a gap where the subject support is received by the subject support receiving portion, the gap being defined by the longitudinal side wall and the longitudinal interior wall.

The gap may be at a location where the subject support emerges from or retracts within the subject support receiving portion as the subject support tilts about the tilt axis.

A radiotherapy device comprising the subject support apparatus of any of the above implementations is also provided.

According to a third aspect, a subject support apparatus for a radiation therapy device is provided, wherein the apparatus comprises a subject support comprising a first longitudinal side wall and a second longitudinal side wall each having a radius of curvature defined with respect to a tilt axis, the subject support having a maximum width less than or equal to double the radius of curvature. The apparatus further comprises a subject support receiving portion comprising a first longitudinal interior wall and a second longitudinal interior wall, each having a radius of curvature defined with respect to the tilt axis, the first longitudinal interior wall and the second longitudinal interior wall being arranged to oppose the first longitudinal side wall and the second longitudinal side wall of the subject support, respectively, wherein the subject support is configured to tilt about the tilt axis relative to the subject support receiving portion, and wherein the radius of curvature of the first and second longitudinal interior walls is greater than the radius of curvature of the first and second longitudinal side walls.

The radius of curvature of the first and second longitudinal interior walls may be less than 8 mm greater than the radius of curvature of the first and second longitudinal side walls.

The subject support receiving portion can be configured such that a tilting of the subject support about the tilt axis causes the first longitudinal side wall to emerge from the subject support receiving portion, and causes the second longitudinal side wall to retract into the subject support receiving portion or, alternatively, causes the second longitudinal side wall to emerge from the subject support receiving portion, and causes the first longitudinal side wall to retract into the subject support receiving portion.

A portion of at least one of the first longitudinal side wall and the second longitudinal side wall May protrude from the subject support receiving portion in all tilt configurations of the subject support.

The portion of at the least one of the first longitudinal side wall and the second longitudinal side wall of the subject support may comprise an additional recess for receiving at least part of an accessory attached to the subject support.

The subject support can comprise a liquid directing feature for channeling fluid away from the subject support receiving portion.

The liquid directing feature may be configured to interrupt a flow of liquid from the subject support towards a gap, the gap being defined by either the first longitudinal side wall and the first longitudinal interior wall, or by the second longitudinal side wall and the second longitudinal interior wall.

The liquid directing feature may be configured to direct the flow of liquid away from the gap.

The liquid directing feature may comprise a lip which protrudes from a surface of the recess.

The liquid directing feature can comprise a channel located within the subject support receiving portion. The channel can be configured to direct liquid towards a longitudinal end of the apparatus. The liquid directing feature may comprise a groove configured to exhibit capillary action.

The apparatus can further comprise a top surface between the first longitudinal side wall and the second longitudinal side wall, the top surface being parallel to the ground when the subject support is in a neutral tilting position, and a base surface between the first longitudinal side wall and the second longitudinal side wall, the base surface being on an opposite side of the subject support as the top surface.

The base surface may be curved with the radius of curvature of the first and second longitudinal walls. The base surface may be substantially flat.

A radiotherapy device comprising the apparatus of any of the above implementations is also provided.

According to a fourth aspect, a subject support apparatus for a radiation therapy device is provided, the apparatus comprising a platform, a subject support movable relative to the platform and supported by the platform, and an interface board movable relative to the platform, wherein the interface board is physically coupled to the subject support such that a translation of the subject support relative to the platform causes a translation of the interface board, and wherein the interface board is configured to electrically connect the subject support to an electrical cable.

The platform may be arranged to support the interface board.

The apparatus may further comprise a slot configured to receive the interface board. The slot may be defined by a gap between the platform and a cover, the cover being disposed generally above the platform.

The cover can be configured to cover an electrical cable.

A movement of the subject support relative to the platform may at least partially expose the slot.

The slot may be configured to accommodate the movement of the interface board relative to the platform.

The interface board can be configured to translate along the slot.

The slot may be less than or equal to 8 mm in length.

The interface board may be mounted to the subject support.

The interface board may comprise a PCB and/or a flat cable. The interface board may further comprise a supporting plate for supporting the PCB and/or the flat cable.

The apparatus may further comprise a cable chain configured to contain an electrical cable. The cable chain may be configured to maintain a plurality of cables. The cable chain may be disposed between a cover and the platform.

The platform can be configured to route one or more electrical cables within the platform.

The interface board may be configured to electrically couple to a tilting module configured to control a tilt of the subject support relative to the platform.

The interface board may be configured to electrically connect the subject support to a plurality of cables.

The interface board can comprise a plurality of electrical connections to the subject support.

A radiotherapy device comprising the apparatus of any of the above implementations is also provided.

According to a fifth aspect, a subject support apparatus for a radiation therapy device is provided, the apparatus comprising a subject support and a subject support receiving portion, wherein the subject support is configured to tilt relative to the subject support receiving portion. The apparatus further comprises a deformable guide comprising a first face and a second face, wherein, throughout a range of tilt configurations, the first face is arranged to be in contact, at least in part, with a longitudinal side wall of the subject support, and the second face is arranged to be in contact with a longitudinal exterior wall of the subject support receiving portion.

The deformable guide may be elastically deformable. The deformable guide may comprise an expanded plastic.

The part of the first face arranged to be in contact with the longitudinal side wall of the subject support can be less than 15 mm in length.

The deformable guide can comprise a low friction coating.

The first face of the deformable guide may comprise a recess.

The deformable guide may comprise a channel extending longitudinally through the deformable guide.

The deformable guide may be in a state of deformation where the subject support is in a neutral tilt position.

The deformation of the deformable guide may increase as an angle between the subject support and the longitudinal exterior wall of the subject support receiving portion decreases.

The deformable guide may expand outwardly from the apparatus as an angle between the subject support and the longitudinal exterior wall of the subject support receiving portion decreases.

The deformable guide can further comprise an upper face, and the subject support may comprise a subject support surface, said subject support surface protruding over the longitudinal wall of the subject support, and the upper face of the deformable guide may be attached to an underside of the subject support surface.

The deformable guide can be attached to the subject support and/or the subject support receiving portion.

The apparatus may comprise a further deformable guide, said deformable guide comprising a third face and a fourth face, wherein, throughout a range of tilt configurations, the third face is arranged to be in contact, at least in part, with a further longitudinal side wall of the subject support and the fourth face is arranged to be in contact with a further longitudinal exterior wall of the subject support receiving portion.

### FIGURES

Specific embodiments are now described, by way of example only, with reference to the drawings, in which:

FIG. 1 depicts a radiotherapy device or apparatus comprising a subject support apparatus according to the present disclosure;

FIGS. 2a-c depict a subject support apparatus in different views, according to the present disclosure;

FIG. 3a depicts a linkage in an extended pose according to the present disclosure;

FIG. 3b depicts a linkage in a retracted pose according to the present disclosure;

FIG. 3c depicts a linkage in an extended pose and a base of a subject support apparatus according to the present disclosure;

FIG. 3d depicts a linkage in an extended pose and a base of a subject support apparatus according to the present disclosure;

FIG. 4a depicts a lower coupling element according to the present disclosure;

FIG. 4b depicts a base for a subject support apparatus according to the present disclosure;

FIG. 5a depicts a base for the subject support apparatus according to the present disclosure;

FIG. 5b depicts a cutaway view of a base for the subject support apparatus according to the present disclosure;

FIGS. 6a-c depict a subject support apparatus according to the present disclosure;

FIGS. 7a-b illustrate a first actuator according to the present disclosure;

FIG. 8a depicts a subject support in a neutral tilt position according to the present disclosure;

FIG. 8b depicts a subject support in a maximally tilted position according to the present disclosure;

FIG. 8c depicts a subject support according to the present disclosure;

FIG. 9 depicts a cross sectional view of a subject support apparatus according to the present disclosure;

FIG. 10 shows a simplified section view of a subject support apparatus according to the present disclosure;

FIG. 11 depicts a simplified section view of an embodiment of a subject support apparatus according to the present disclosure;

FIG. 12 depicts a simplified section view of an embodiment of a subject support apparatus according to the present disclosure;

FIG. 13 depicts a simplified section view of an embodiment of a subject support apparatus according to the present disclosure;

FIGS. 14a-b depict a simplified section view of an embodiment of the subject support apparatus of the present disclosure;

FIG. 15 depicts a number of simplified section views of a deformable guide according to the present disclosure;

FIGS. 16a-d illustrate a subject support apparatus according to the present disclosure;

FIG. 17 depicts a subject support apparatus according to the present disclosure; and

FIGS. 18a-c depict views of a movable connection between a longitudinal sledge and a platform according to the present disclosure.

### LIST OF REFERENCES

100 Radiotherapy device
106 Radiation source
111 Vertical z-axis
113 Longitudinal y-axis
114 Subject support apparatus
115 Transverse x-axis
116 Gantry
117 Gantry rotation mechanism
118 Base
120 Rotation mechanism
124 Isocenter
140 Patient
210 Upper unit
211 Tilt axis
212 Subject support
213 First surface
214 Platform
215 Upper lateral sledge
216 Longitudinal sledge
217 Lower lateral sledge
218 Longitudinal groove
219 Longitudinal ridge
220 Linkage
222 First leg
224 First support element
226 Second leg
227 Second support element
228 Base
229 Lower coupling element
230 Actuation mechanism
232 Drive member
245 Skirt
314 Subject support apparatus
321 Attachment element
350 Recessed portion
361a,b First coupling features
370 Second coupling feature
371 Second coupling feature
375 Mounting feature
381 Gap
429 Lower coupling element
450 Recess
453 Coupling features
550 Hole
621 First guide
622 First guide
623 First guide
624 First guide
625 First actuator
635 First guide block
636 First guide block
637 First guide block
638 First guide block
639 Rotational coupling
640 Plate
645 Motion converter
772 Electric motor
774 Belt and pulley arrangement
776 Screw
800 Subject support apparatus
810 Subject support
811 First surface
812 Central longitudinal axis
813 Tilt axis
815 Lower surface
816 Longitudinal sledge
817 First longitudinal side wall
819 Extending edge
820 Subject support receiving portion
821 Side portion
823 First longitudinal exterior wall
825 First longitudinal interior wall
831 Gap
833 Cutout region
835 End portion
837 Groove
841 Region
1000 Subject support apparatus
1010 Subject support
1011 First surface
1012 Central longitudinal axis
1013 Tilt axis
1016 Longitudinal sledge
1017 Longitudinal side wall
1019 Edge portion
1020 Subject support receiving portion
1021 Side portion
1023 Longitudinal interior wall
1027 Flattened portion
1031 Gap
1041 Cutaway region
1118 Protruding lip
1224 Lip
1226 Lower surface
1317 Side surface
1320 Cavity
1328 Insert
1400 Subject support apparatus
1415 Exterior face
1418 Deformable guide
1424 Longitudinal exterior wall
1431 First face
1433 Second face
1435 Upper face
1437 Channel
1500 Subject support apparatus
1514 Cover
1516 Longitudinal sledge
1518 Platform
1544 Lower lateral sledge
1560 Guide rails
1562 Cable chain
1646 Interface board
1848 Electrical connection points
1850 Supporting plate

### DETAILED DESCRIPTION

### Radiotherapy Device—Overview

Arrangements of the present disclosure may be implemented to any suitable or desirable radiotherapy device, for example a treatment device and/or an imaging device.

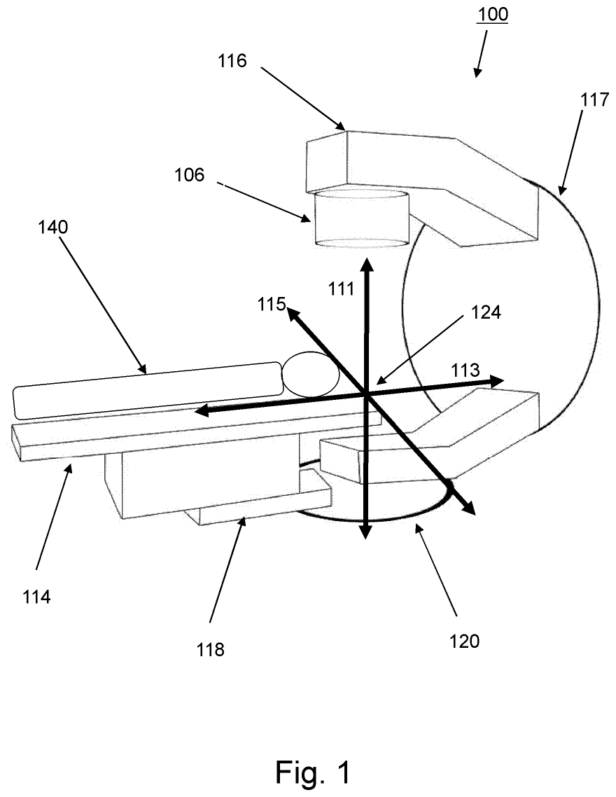

For example, arrangements of the present disclosure may be implemented to the radiotherapy device 100 shown in FIG. 1. FIG. 1 shows an example of a radiotherapy device 100 that combines the rotation of the patient with rotation of the radiation source. The radiotherapy device includes a subject support apparatus 114 for supporting a subject, or patient 140, and a radiation source 106 which is supported by a gantry 116. In the present arrangement, the subject support apparatus 114 is configured to be rotatable around the vertical z-axis 111, while the gantry 116 is configured to be rotatable about the longitudinal y-axis 113 around the subject support apparatus 114. The gantry 116 is a C-arm gantry or open gantry. A gantry rotation mechanism 117 rotates the gantry 116 about the y-axis 113. The radiation source 106 is configured to emit radiation along a radiation axis. Due to rotation of the gantry 116, radiation can be delivered to the patient 140 from a plurality of angles in a radiation plane.

In the example of FIG. 1, a rotation mechanism 120, disposed under the gantry 116, is provided to rotate the subject support apparatus 114 about an axis of rotation of the subject support apparatus 114 in the radiation plane that coincides with the z-axis 111. In particular, the axis of rotation 111 of the subject support apparatus coincides with the isocenter 124 of the radiotherapy device, such that the subject support apparatus 114 rotates about the isocenter 124. A gantry rotation mechanism 117 is provided for the gantry 116 disposed opposite the subject support apparatus 114 with respect to the axis of rotation 111. In the following discussion, the vertical z-axis 111 is taken to be the axis perpendicular to the plane of the subject support apparatus when it is in its neutral position (parallel to the plane of the floor), the transverse x-axis 115 is taken to be the transverse axis (short side) of the subject support apparatus 114, and the longitudinal y-axis 113 is taken to be the longitudinal axis (long side) of the subject support apparatus. In the example of FIG. 1, the axis of rotation of the subject support apparatus 114 coincides with the vertical z-axis 111.

The subject support apparatus 114 includes a base 118 which supports the subject support apparatus 114 and which connects the subject support apparatus 114 to the rotation mechanism 120.

The subject support apparatuses described herein may be used in conjunction with a medical device, for example an imaging device. In a preferred implementation, the medical device is a radiotherapy device.

### Subject Support Apparatus—Overview

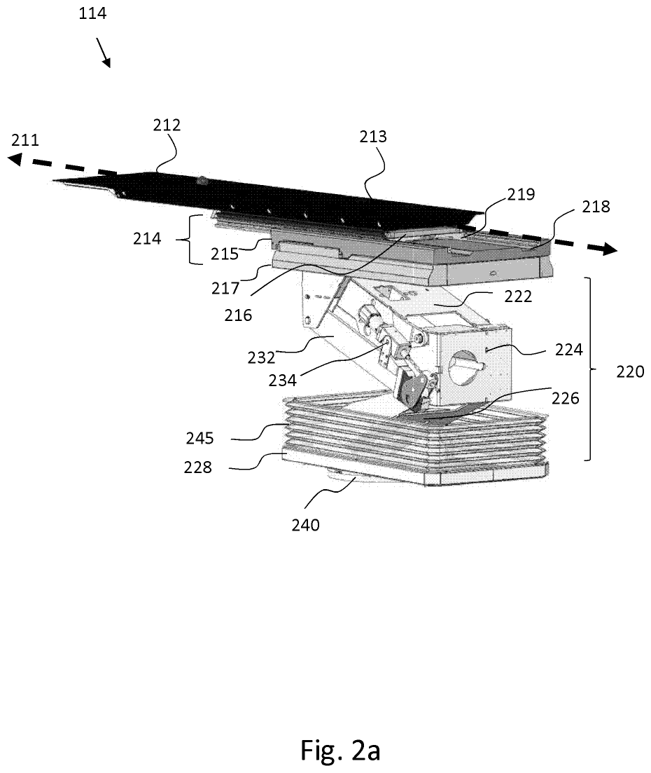

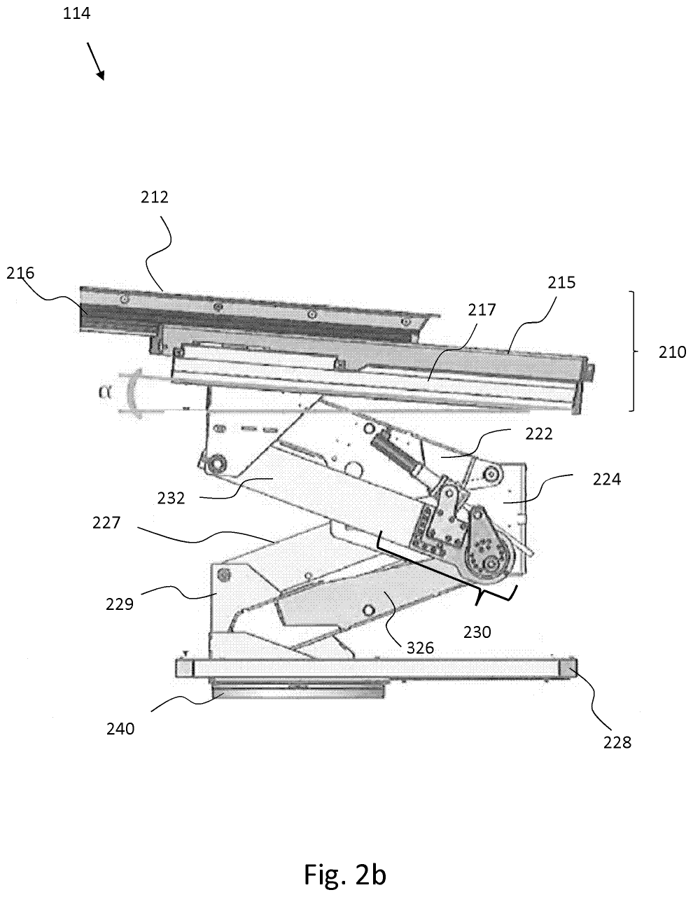

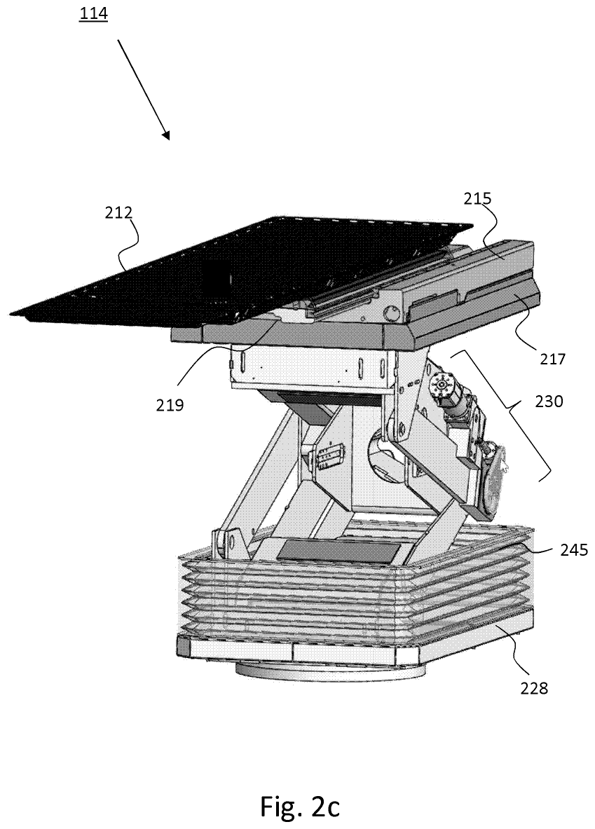

FIGS. 2a, 2b, and 2c depict the subject support apparatus 114 in more detail. FIG. 2a depicts an angled rear view of the subject support apparatus 114. FIG. 2b depicts a side-on view of the subject support apparatus 114. FIG. 2c depicts an angled front view of the subject support apparatus 114.

The subject support apparatus 114 is configured to move and/or position the subject or patient 140. The subject support apparatus 114 may be configured to move the patient 140 in any, some or all (e.g. a combination) of three translation degrees of freedom: height (e.g. parallel to the z-axis 111), longitudinal movement (e.g. parallel to y-axis 113) and a lateral movement (e.g. parallel to the transverse x-axis 115). Furthermore, the subject support apparatus 114 may be configured to move the patient 140 in any, all, or a combination of three rotational degrees of freedom: roll (e.g. around the longitudinal y-axis 113), pitch (e.g. around the x-axis 115) and yaw (e.g. around the z-axis 111).

The subject support apparatus 114 comprises a subject support 212 (also referred to as a tabletop or couch), a platform 214, a linkage 220 (also referred to as a support structure or arm) and a base 118. In some embodiments, the subject support apparatus 114 may comprise a subject support receiving portion. In some embodiments, the platform 214 may comprise the subject support receiving portion, e.g. in an integrated form. Throughout this description, it is therefore to be understood that a subject support receiving portion may be a platform (e.g. platform 214), or an additional component, e.g. arranged between the platform 214 and the subject support 212.

The subject support 212 is configured to support a patient, or subject, on a first surface 213 (also referred to as a patient positioning surface or subject support surface). The subject support 212 is supported directly or indirectly by the platform 214, and is tiltable relative to the platform along a tilt axis 211. The tilt axis may correspond to a roll axis. In some embodiments, the subject support 212 is disposed on or in a subject support receiving portion, which may be the platform 214, a part of the platform 214, or an external component attached or coupled to the platform 214. In some embodiments, the tilt axis 211 may be the longitudinal axis 113 or an axis parallel to the longitudinal axis 113. That is, the tilt axis 211 may be a central longitudinal axis 113 along the surface of the subject support 212.

The platform 214 is supported by the linkage 220, which is configured to support and move the platform 214 and the subject support 212 above a floor, such as the floor of a treatment room. The platform 214 is connected to a base 228, such as base 118, which supports the subject support apparatus 114 and which connects the subject support apparatus to the rotation mechanism 120. In some implementations, the base 228 may connect to the floor directly, without an intervening rotation mechanism 120. The base 228 may be embeddable in the floor.

The subject support 212 and platform 214 may, in combination, be referred to as an upper unit 210. In use, the patient 140 may lie on the subject support 212. In other words, in use, the patient 140 may contact the first surface 213 of the subject support 212.

It will be appreciated from the description that the subject support apparatus 114 enables movement of the subject support 212 in one or more degrees of freedom. In some implementations, the subject support apparatus 114 only provides movement of the subject support 212 in one degree of freedom, a subset of the degrees of freedom listed, or all degrees of freedom disclosed in this application.

The subject support 212 can be moved linearly with respect to the platform 214 along the tilt axis 211 (or an axis parallel to the tilt axis 211). This movement may be controlled via a linear actuator. This linear movement may be described as a translation. In other words, the subject support 212 is configured to translate linearly with respect to the platform 214.

### Platform of Subject Support Apparatus

The platform 214 comprises a longitudinal sledge 216, an upper lateral sledge 215, and a lower lateral sledge 217. The longitudinal sledge 216 is movably coupled to the subject support 212, and supports the subject support 212. For example, the longitudinal sledge 216 is coupled to the subject support 212 via a tilting module.

The longitudinal sledge 216 comprises a longitudinal ridge 219, which is configured to be received by, or to couple with, a corresponding longitudinal groove 218 on the upper lateral sledge 215, on the underside of the longitudinal sledge 216. The platform 214, specifically the upper lateral sledge 215, comprises the corresponding longitudinal groove 218. The longitudinal groove 218 is formed on an upper surface of the upper lateral sledge 215. The longitudinal ridge 219 is received by the longitudinal groove 218 in order to couple the subject support 212 to the upper lateral sledge 215, so as to allow movement of the subject support 212 with respect to the platform 214 along an axis defined by the longitudinal groove—for example along tilt axis 211. Movement of the subject support 212 is controlled via a linear actuator, such that the subject support 212 is configured to move in a longitudinal, linear manner with respect to the platform 214 and the other components of the subject support apparatus 114. In some embodiments, the platform 214 and the other components of the subject support apparatus (e.g. other than the subject support 212) may be collectively referred to as the patient support base.

It will be appreciated that the subject support 212 and the platform 214 may be coupled in any way which allows movement, for example translational movement. For example, the ridge and groove arrangement may be swapped in some implementations such that the longitudinal sledge 216 comprises the groove and the upper lateral sledge 215 comprises the corresponding ridge.

The upper lateral sledge 215 is coupled to the lower lateral sledge 217 in a movable arrangement, such that the upper lateral sledge 215 can move relative to the lower lateral sledge 217, for example in a lateral direction which is perpendicular to the longitudinal direction.

### Rotation Mechanism for Adjusting Pitch

The linkage 220 is configured to adjust the height of the upper unit 210, and also acts as a rotation mechanism to rotate the upper unit 210—for example rotating the upper unit 210 to change its pitch. The rotation is made with respect to a horizontal plane, and/or with respect to the floor of the treatment room. In FIG. 2b, a pitch angle is marked a.

In the implementation depicted in FIGS. 2 a-c, the rotation mechanism of the linkage 220 comprises a drive member 232 and an actuation mechanism 230. The rotation mechanism of the linkage 220 is configured to impart a force, via the drive member 232, to an underside of the platform 214 to thereby rotate the platform 214 (and the connected subject support 212) with respect to the horizontal plane.

The rotation mechanism is configured to rotate the upper unit 210, i.e. both the subject support 212 and the platform 214. Because the rotation mechanism of the linkage 220 is positioned underneath the upper unit 210, rather than forming part of the upper unit 210 as in known designs, the weight and size of the upper unit 210 can be significantly reduced. Because the rotation mechanism does not form part of the upper unit 210, it is not necessary to translate the entire upper unit 210 (including the rotation mechanism) as part of the linear translation of the subject support 212.

### Linkage-Upper and Lower Elements

The linkage 220 is configured to bear the weight of the upper unit 210, as well as a patient positioned on the subject support 212. Multiple implementations of the linkage 220 are envisaged. In the implementation depicted in FIGS. 2a, 2b and 2c, the linkage 220 comprises an upper element (or member) coupled to an underside of the upper unit 210, and a lower element (or member) coupled to the base. The upper element may comprise a first leg 222 and the lower element may comprise a second leg 226 coupled to the base. The first leg 222 and second leg 226 are coupled to one another via a first support element 224. The first support element 224 may be referred to as a support block or anchor element herein.

In this implementation, the linkage includes a second support element 227 which forms part of the lower element. The second support element 227 is coupled to the second leg 226 in a parallelogram four-bar linkage, enabling the orientation of the first support element 224 to remain the same during motion.

In some implementations, the upper unit 210 is rotationally coupled to the linkage 220 to allow rotation about a rotation axis. In a simple implementation, the linkage 220 may be coupled to the upper unit 210 via the interaction between a shaft and one or more bearings which receive the shaft. For example, the one or more bearings may be mounted to an underside of the upper unit 210, and configured to receive a shaft. For example, an upper region of the first leg 222 may culminate in a double-ended shaft, with each end of the shaft being received in a bearing mounted to a base of the upper unit 210. In this implementation, the orientation of the shaft and bearings defines an axis of rotation about which the upper unit 210 may rotate with respect to the linkage 220. Other implementations include a ball-joint, or any other mechanical connection that allows rotation of the upper unit 210 with respect to a rotation axis.

The second leg 226 is coupled to the base. The coupling may be achieved via a lower coupling element 229 and the second leg 226 may be configured to rotate with respect to the lower coupling element as part of a height adjustment mechanism. The lower coupling element 229 extends upward out of the plane of the base, allowing the second leg 226 to be coupled to the lower coupling element 229 to define a rotation axis parallel with the rotation axis about which the upper unit 210 rotates with respect to the first leg 222.

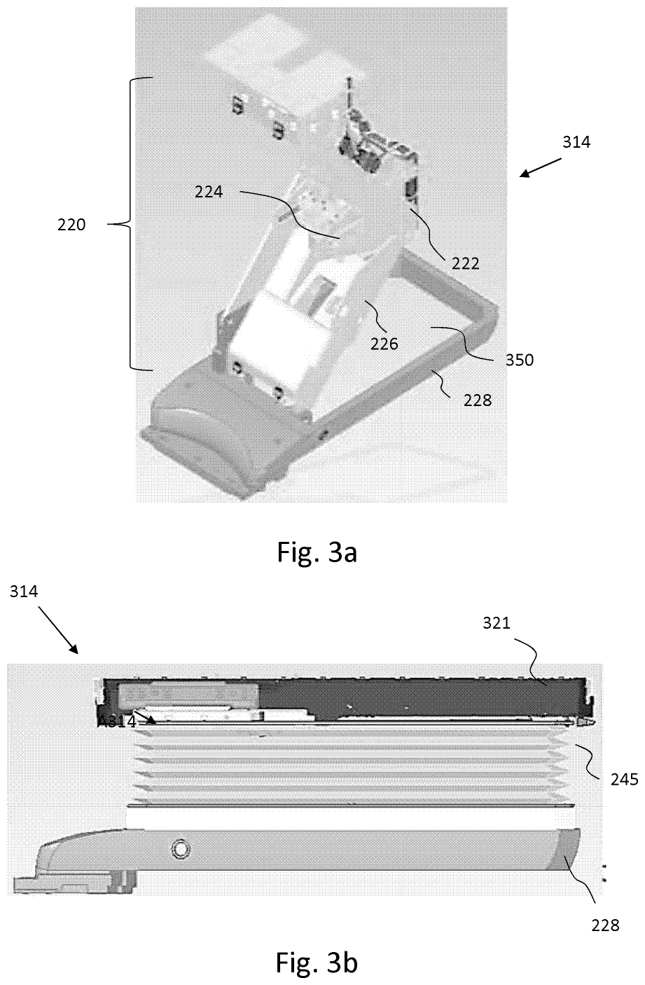

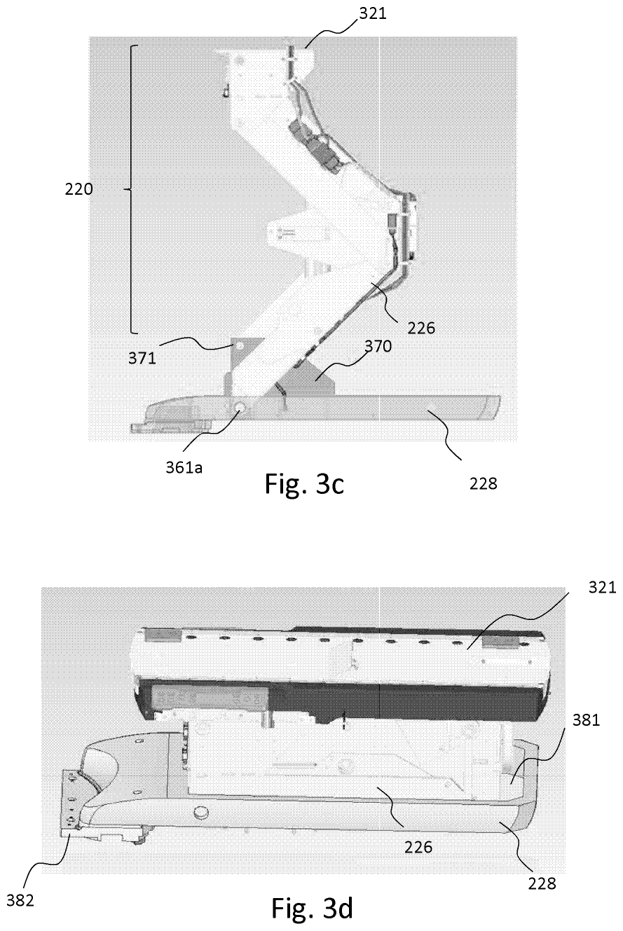

Implementations of the base 218 and the linkage 220 will be described further with reference to FIGS. 3-5.

### Height Adjustment Mechanism

The linkage 220 may also comprise a height adjustment mechanism (not shown in the figures). The height adjustment mechanism is configured to adjust the height, i.e. vertical distance, of the upper unit 210 above the floor or base. The height adjustment mechanism comprises one or more motor mechanisms. An upper motor mechanism may be positioned within, form part of, or be coupled to, the first support element 224. A lower motor mechanism may be positioned within, form part of, or be coupled to, the lower coupling element 229.

The height adjustment mechanism may be formed by one or multiple different mechanisms. The height adjustment mechanism may be configured to adjust the vertical distance between the first support element 224 and the upper unit 210 by actuating the first leg 222. Thereby, the height of the upper unit 210 above the floor is increased. The height adjustment mechanism comprises a rotational mechanism or motor configured to produce a rotary motion of the first leg 222 with respect to the first support element 224. This may be a rotary hydraulic motor. This rotary motor is housed within the first support element 224.

Optionally, an additional rotary motor may be provided. This rotary motor may be referred to as a lower rotary motor in contrast to the rotary motor described above, which may be referred to as the upper rotary motor. The lower rotary motor is also housed within the first support element 224 and is configured to drive rotation with respect to the first support element 224 and the second leg 226. The height adjustment mechanism may thereby also be configured to adjust the vertical distance between the first support element 224 and the base 228 and/or floor of the treatment room, by actuating the second leg 226 using this lower rotary motor. Thereby, the height of the upper unit 210 is adjusted. By synchronously driving rotation using both the upper and the lower rotary motor, the vertical height of the subject support 212 may be adjusted.

For example, the height adjustment mechanism may comprise a lower rotational mechanism or motor, e.g. a rotary hydraulic motor, configured to produce a rotary motion of the second leg 226 with respect to the first support element 224.

The height adjustment mechanism is configured to control a height of the upper unit 210 above the floor of the treatment room. As described above, the upper unit 210, and in particular the base of the upper unit 210, is rotationally coupled to the linkage 220 to allow rotation about a rotation axis. By adjusting the height of the upper unit 210 above the floor of the treatment room using the height adjustment mechanism, the height of this rotation axis can also be adjusted.

While a linkage 220 and height adjustment mechanism has been described which comprises a mechanism capable of rotating one or a plurality of supporting legs about rotation axes in order to adjust the height of the upper unit 210, the height adjustment mechanism may take multiple forms.

For example, the height adjustment mechanism may comprise an arrangement of hydraulic pistons positioned and configured to adjust the height of the upper unit 210. An alternative implementation may involve a scissor lift mechanism. The skilled person will be aware of other possible ways in which the height of a upper unit 210 may be adjusted. Regardless of the specific implementation of the linkage 220 and/or height adjustment mechanism, the rotation mechanism 120 is coupled to the linkage 220 and is configured to impart a force to an underside of the upper unit 210 in order to rotate the upper unit 210 with respect to the linkage 220.

In some implementations, the subject support apparatus 114 also comprises a skirt 245 (also referred to as bellows) configured to cover the linkage 220. The skirt 245 is connectable between the base 228 and the upper unit 210. The skirt 245 has a flexibility, and in particular may have a concertina configuration, i.e. be configured to extend, compress, or collapse in folds like those of a concertina. Thus, patients and clinicians are protected from injury by virtue of the moving mechanisms described herein. It is simpler to provide this protection using a simple skirt 245 by virtue of the present design, and in particular by virtue of the rotation mechanism being attached to and supported by the support structure. In FIGS. 2a and 2c, the skirt is folded or compressed down away from the upper unit 210 so that the linkage 220 and rotation mechanism can be seen.

### Extended and Retracted Pose

FIGS. 3a and 3b illustrate an implementation of a subject support apparatus 314, e.g. subject support apparatus 114, in which some elements have been omitted for simplicity. FIG. 3a shows the subject support apparatus 314 in an extended pose while FIG. 3b shows the subject support apparatus 314 in a retracted pose.

The subject support apparatus 314 comprises a linkage 220, which includes a first member 222 (also referred to as an upper element or first leg), and a second member 226 (also referred to as a lower element or second leg). The first member 222 can be coupled to a platform, for example the platform 214 described above, e.g. by means of an attachment element 321.

The first member 222 is rotatably coupled to the second member 226 via a first support element 224. The first support element 224 may be a component of the first member 222 or the second member 226, or may be a separate component.

The second member 226 is rotatably coupled to a base 228. In this implementation, the second member 226 is directly connected to the base 228, although in some implementations the second member 226 may be connected to the base 228 via an intermediary component.

The base 228 comprises a recessed portion 350, arranged such that it can receive at least a portion of the linkage 220. Such a recessed portion 350 may comprise a recess that extends through the base 228, as in the implementation shown in FIG. 3a. In other words, the recessed portion 350 may be a hole or cut-out of the base 228. In some implementations, the recessed portion 350 may be a hole or cut-out surrounded on all sides by walls of the base 228, e.g. a depth of the recessed portion 350 may be equal to the thickness of the base 228. In other implementations, the recessed portion 350 may be bordered on two or three sides of the hole or cut-out, such that one side of the recessed portion is left open. Other implementations may comprise a recess that partially extends into the base, such as to accommodate at least a portion of the linkage 220. In other words, in some implementations, the recessed portion 350 may have a thickness less than that of the rest of the base 228. In some implementations, the recess, e.g. the recessed portion 350, may be surrounded by the rest of the base 228, such that the recessed portion 350 is contained within the base 350. That is, a depth of the recessed portion 350 may be less than the thickness of the base 228. In some embodiments, the recessed portion 350 extends to one or more edges or sides of the base 350. In some implementations, the recessed portion 350 has a length and width equal to or larger than that of the lower element of the linkage 220. In some implementations, the recessed portion 350 has a length which is less than the length of the lower element of the linkage 220, and at least one side of the recessed portion 350 is not bordered by a thicker portion of the base 228.

In the extended pose of FIG. 3a, the linkage 220 extends beyond, e.g. above, the base 228. That is, in the extended pose, the linkage 220 may protrude from the base 228. In the retracted pose of FIG. 3b, the linkage 220 is at least partially received by the base 228, e.g. by the recessed portion 350. That is, in the retracted pose, at least some of the linkage 220 has a height above the ground which is less than the thickness of the base 228. In some implementations, at least part of the thickness of the second member 226 is received within the base 228, e.g. within the recessed portion 350.

In some implementations, in the retracted pose, the members of the linkage 220 may collapse, e.g. fold. For example, the second member 226 may, in the retracted pose, be substantially parallel to the ground. In some implementations, the members of the linkage 220 may collapse such that each member of the linkage 220 is substantially parallel to the ground, e.g. flat, when in the retracted pose.

The linkage 220 can be configured to adjust at least one of height, pitch, roll and yaw of a platform coupled to the first member 222 of the linkage 220. In order to accommodate such adjustments, the linkage 220 can maintain a number of intermediate poses between the extended pose of FIG. 3a and the retracted pose of FIG. 3b. In such intermediate poses, the linkage 220 may extend partially beyond the base 228, with a portion of the linkage 220 remaining received by the recessed portion of the base 228.

In some implementations, a skirt 245 is provided. Skirt 245 is connectable between the base A228 and an attachment element 321 (e.g. for a platform). The skirt 245 has a flexibility, and in particular may have a concertina configuration, i.e. be configured to extend, compress, or collapse in folds like those of a concertina. It can be seen that in the retracted pose in FIG. 3b, the skirt 245 has collapsed into folds in order to accommodate the retraction of the linkage 220 into the base 228. The skirt 245 may be used to ensure that cables, fingers, clothing or the like do not get trapped by the linkage 220 when the linkage 220 moves between the extended pose and the retracted pose. It is to be understood that the skirt 245 is an optional feature.

The portion of the linkage 220 received by the base 228 when in the retracted position shown in FIG. 3b may comprise a portion of the second member 226, and may extend along the length of the second member 226. This may include the second member 226 being received by the base 228 in its entirety. For example, the depth of the recessed portion 350 may be greater than or equal to the thickness of the second member 226. In such an implementation, the first member 222 extends beyond the recessed portion 350 of the base 228. In some implementations, the depth of the recessed portion 350 may be less than the thickness of the second member 226, and the second member 226 may protrude from the recessed portion 350 when in the retracted pose.

In other implementations, the portion of the linkage 220 received by the base 228 when in the retracted position shown in FIG. 3b may comprise at least a portion of the first member 222, and may extend along the length of the first member 222. In such an implementation, the second member 226 is received by the base 228 in its entirety when in the retracted pose.

The recessed portion 350 of the base 228 can be configured to receive the entirety of the linkage 220. In such implementations, the skirt 245 will collapse into folds such that the skirt 245 is substantially flat. Platform 214 may then rest on the upper surface of the base 228, and may be connected to the linkage 220 via an attachment element 321. In such implementations, the depth of the recessed portion 350 may be at least the thickness of the linkage when the linkage is in the retracted pose. The thickness of the linkage when the linkage is in the retracted pose may be, for example, a sum of the thicknesses of each of the members making up the linkage.

In some implementations, one or more members of the linkage 220 may be received within the recessed portion 350 when the linkage is in the retracted pose, and at least one further member of the linkage 220 may protrude, e.g. extend, from the base 228.

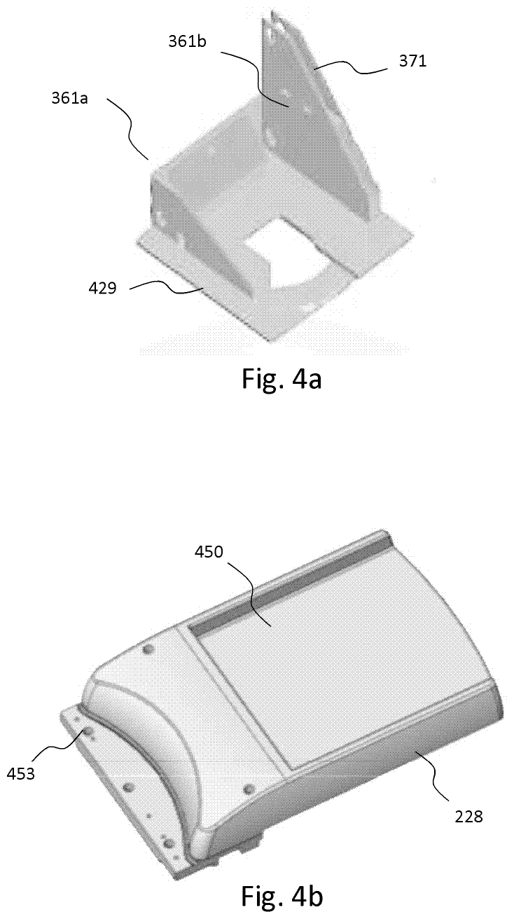

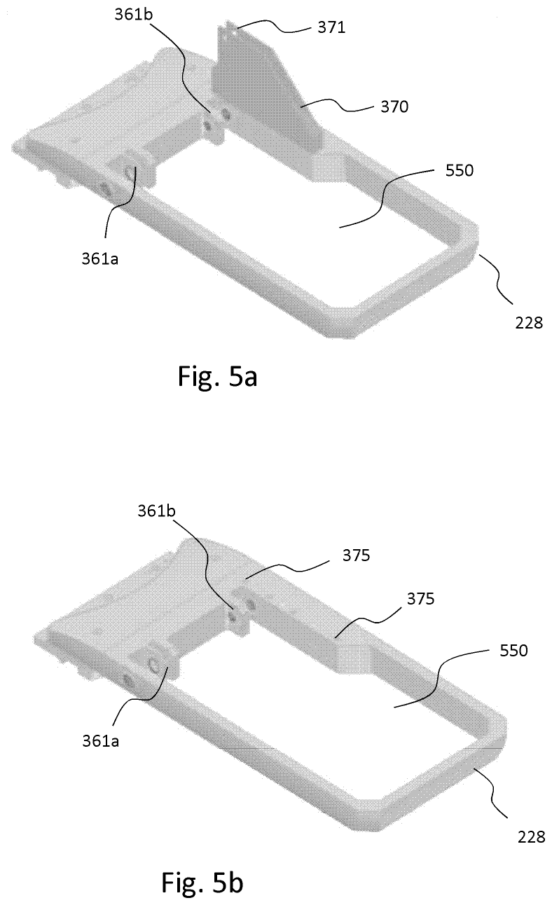

FIGS. 3c and 3d illustrate an embodiment of a subject support apparatus 314 in which some elements have been omitted for simplicity. In FIG. 3c, the linkage 220 can be seen in an extracted pose, and in FIG. 3d the linkage can be seen in a retracted pose. As shown in FIG. 3d, the base 228 may extend beyond the length of the second member 226 when the linkage 220 is in the retracted pose. In other words, the recessed portion 350 of the base 228 may be longer than the length of the linkage 220 in the retracted pose, e.g. the length of the second member 226. A gap or space 381 may be formed when the linkage 220 is in the retracted pose. Such a space can be used, for example, for the storage of electrical components which may be attached to the linkage 220 or other parts of the apparatus. Base 228 may be coupled, e.g. attached, to the floor via attachment means 382, such as one or more of screws, bolts, axles, hinges, or the like. In some implementations, the attachments means 328 may allow for the base 228 to rotate about a vertical axis relative to the floor. FIGS. 3c and 3d further illustrate a first coupling feature 361 and second coupling features 370 and 371. These features will be described further with reference to FIGS. 4a-5b.

### Base and Lower Coupling Element

FIG. 4a shows a lower coupling element in more detail. The lower coupling element comprises first coupling features 361a and 361b configured to allow the second leg 226 to be coupled to the lower coupling element 429. The lower coupling element 429, e.g. lower coupling element 229, also includes a second coupling feature 371 which is configured to allow the second support element 227 to be coupled to the lower coupling element 429. The lower coupling element 429 can be connectably coupled to a base, or may form part of a base. Features of the such a base may have equivalent or similar properties to the corresponding features of the base 228 described above. The inclusion of such a component for coupling the base 228 to the linkage 220 decreases the minimum height of the platform when the linkage is in the retracted position. That is, the use of a component such as lower coupling element 429 decreases the minimum step-on height of the platform, making it easier for patients to get onto the subject support for their scan or treatment.

FIG. 4b shows an embodiment of a base 228 of the subject support apparatus. The base 228 includes a recess 450 which is configured to receive a portion of the linkage 220 when the linkage is in a retracted pose. Recess 450 may be an example of recessed portion 350. Where the linkage 220 comprises the second leg 226, such a retracted pose may involve the recess 450 receiving at least a portion of a second leg 226 of the linkage 220, where the received portion extends along the length of the second leg 226. In such a pose, the second leg of the linkage 220 may partially or fully extend beyond the recess 450. In the embodiment shown in FIG. 5b, the recess is configured to receive at least part of the second leg 226 when the linkage is in the retracted pose, and the recess 450 extends partially into the base 228.

That is, in the implementation illustrated in FIG. 4b, the depth of the recess 450 is less than the thickness of an area of the base 228 surrounding (e.g. on two, three or four sides of the recess 450) or defining (e.g. lining, or bordering) the recess 450, e.g. an outer wall of the base 228.

In some implementations, an outer edge of the base 228 is also recessed, as illustrated in FIG. 4b. In such implementations, the recessed portion 350 may extend to an outer edge of the base 228. In some such implementations, the length of the recessed portion 350 may be less than the length of the linkage 220 when in the retracted pose, or less than the length of a member (e.g. second member 226) of the linkage 220. In the retracted pose, a portion of the length of the linkage 220 may be received by the recessed portion 350, and a remaining portion of the length of the linkage 220 may protrude or extend laterally, overhanging the base 228.

The base 228 further comprises a plurality of coupling features 453, e.g. attachment means 328, which allow for the base 228 to be movably coupled to the a surface disposed below, for example the floor of a treatment room. The plurality of coupling features 453 may take the form of a plurality of holes, configured such that the base 228 can be coupled to the surface disposed below.

FIG. 5a depicts the base 228 of the subject support apparatus 314. The base 228 comprises a recessed portion 350, which is configured to receive at least a portion of a linkage 220. In this embodiment, the recessed portion 350 extends through the base A228, such that a significant amount, for example at least a quarter, a third, or a half, of the linkage 220 or of a member of the linkage 220, may be received by the recessed portion 350. That is, the recessed portion 350 may comprise a hole 550 dimensioned to receive at least a portion of a member of the linkage 220. The depth of the recessed portion 350 may be equal to the thickness of the base 228, or to an area of the base 228 surrounding the recessed portion 350. For example, the recessed portion 350 may be surrounded by a wall of the base 228. The recessed portion 350 is shaped such that it may receive the portion of the linkage 220 where the received portion extends along the length of the linkage 220. That is, the length of the recessed portion 350 may be at least a length of a member of the linkage 220, or at least a length of the linkage 220 when the linkage 220 is in the retracted pose. In other words, the length of the linkage 220 may be a longitudinal length of the linkage 220 when the linkage 220 is in the retracted pose, e.g. along a longitudinal axis of the second member 226. The width of the recessed portion 350 may be at least a width of a member of the linkage 220, or at least a width of the linkage 220.

In this implementation, the base A228 comprises at least one coupling element which allow for the base A228 to connectably and movably couple to the linkage A220. First coupling features A361a and A361b are configured to allow the linkage A220 to be coupled to the lower coupling element. In particular, the first coupling features A361a and A361b can be utilised to allow the second leg A226 of the linkage A220 to be coupled to the base A228. These coupling features A361a, A361b can be arranged within the recessed portion 350 such that the linkage A220 can be fully or partially received into the base A228. Although two coupling features are shown in FIGS. 5a and 5b, this is merely exemplary. In some implementations, a single coupling feature may be used, such as a centrally positioned hinge or the like. In some implementations, more than two coupling features may be used.

Second coupling features 370 and 371 allow a supporting element (not shown) of the linkage 220 to be coupled to the base 228. The second coupling features 370 and 371 may comprise a raised slot 370 with a number of holes 371 through which the second support element 227 can be coupled to the base, as illustrated in FIG. 5a. Such a slot may be configured, e.g. dimensioned and/or positioned, such that it can partially or fully receive the second support element 227 when the linkage 220 is in a retracted position.

Such coupling features may allow for the linkage 220 to be movably or rotatably coupled to the base 228.

The plurality of coupling features 361a, 361b, and optionally 370, 371, may form part of the base 228, which may allow the base 228 to be more compact than in previously known systems. For example, the arrangement of coupling features within the recessed portion 350 allows for a decrease in the height of the base, and/or a decrease in the minimum, or step-on, height of the platform.

FIG. 5b shows a cutaway view of the base 228 for the subject support apparatus 314. Mounting features 375 are visible on the upper surface of the base 228, disposed generally beneath the raised slot 370 (not shown in this figure). Such mounting features may be used to mount the raised slot 370 to the base 228, or alternatively the second support element 227 may be directly connected to the base using the mounting features 228. The mounting features 375 may take the form of a plurality of holes, dimensioned to receive one of the raised slot 370 or the second support element 227 of the linkage 220. In some implementations, the raised slot 370 may be fused to the base 228, or the base 228 may comprise the raised slot 370.

It is to be understood that the raised slot 370 is an optional feature.

The base 228, as depicted in any of the embodiments disclosed herein, can be manufactured via a casting process or the like. Such a manufacturing process is less expensive than alternatives like welding, thus the base 228 offers a less costly alternative to that of known systems. It is possible to manufacture base 228 using a casting process as the coupling elements for coupling the base to the linkage 220 form part of the base A228. Casting the base 228 may involve merging some of the function of a height adjustment unit with the base 228, for example through a connection with a supporting element via a raised slot A370. A base 228 formed via a casting process may also provide increased stiffness and rigidity, and may provide improved support to the linkage 220 and other components of the apparatus.

The inclusion of the coupling elements in the base 228 also allows a reduction in the step-on height of the subject support apparatus. Typically, coupling arrangements between a standard, non-recessed base (as would be found in known designs) and a linkage comprise a connecting element which sits atop the base, such that the linkage is movable about a point atop the base adding to the step-on height of the apparatus. Such a connecting element may be separately manufactured, and the components would then have to be welded (for example) together, which makes such an arrangement costly and thus adds a further disadvantage to known designs.

### Tilting Arrangement

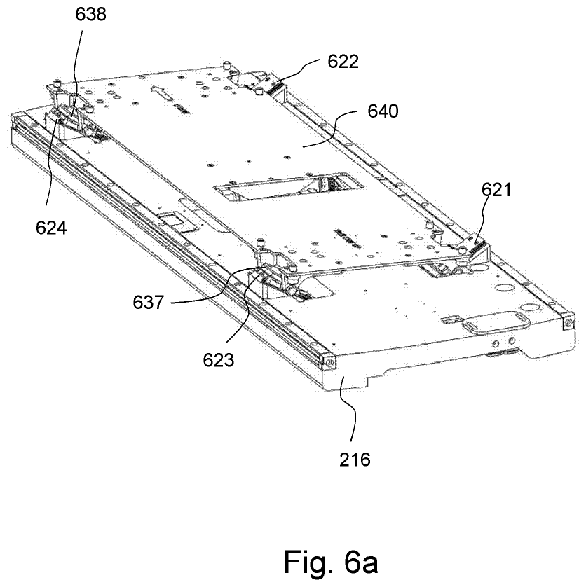

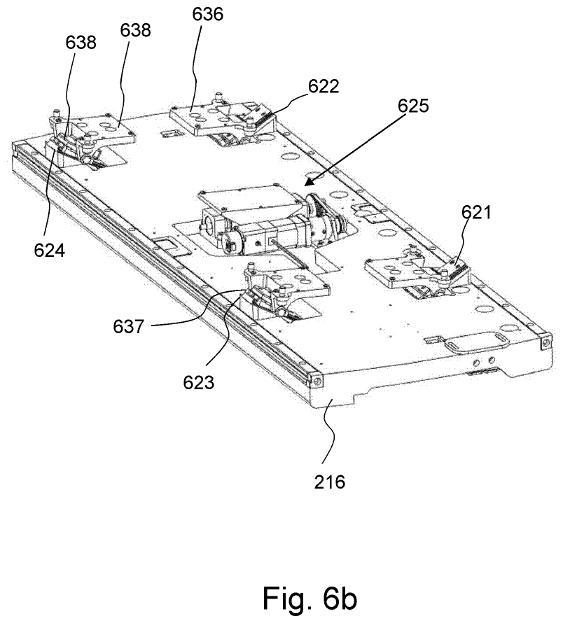

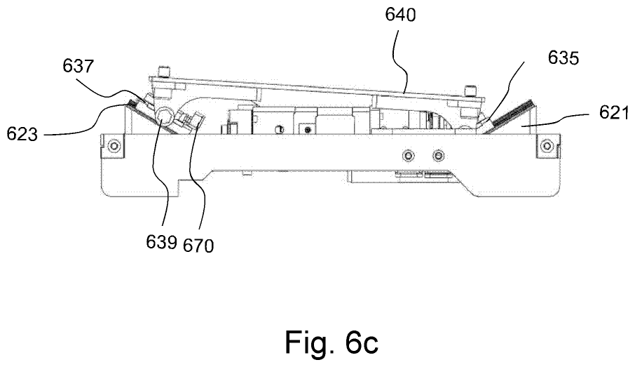

FIGS. 6a-c depict an exemplary implementation of the subject support apparatus 114 with a number of elements excluded in order to better depict the longitudinal sledge 216 (also referred to as a first platform element).

FIG. 6a is an angled view depicting the longitudinal sledge 216 coupled to a plate 640. FIG. 6b depicts the same angled view but with the plate 640 removed so as to better depict the arrangement of a first actuator 625. FIG. 6c depicts a front view of the same arrangement as FIG. 6a.

When fully assembled with the subject support 212 coupled to the plate 640, the subject support 212 is tiltable with respect to the platform 214. The subject support 212 is tiltable about the tilt axis 211, which may be a fixed axis. The tilt axis 211 is parallel with and/or may be coincident with the longitudinal centre line of the subject support and/or the longitudinal centre line of the plate 640 and/or the longitudinal centre line of the longitudinal sledge 216. That is, the longitudinal centre line of the subject support may be coincident with the longitudinal centre line of the plate 640 and the longitudinal centre line of the longitudinal sledge 216.

In the depicted arrangement, the subject support apparatus 114 further comprises first guides 621, 622, 623, 624 disposed on the longitudinal sledge 216. The first guides 621, 622, 623, 624 are inclined with respect to the longitudinal sledge 216. The first guides 621, 622, 623, 624 are fixed with respect to the longitudinal sledge 216 and extend into recesses in the longitudinal sledge 216. By providing inclined guides 621, 622, 623, 624 which extend into recesses in the base, a gap between the longitudinal sledge 216 and a subject support surface can be advantageously reduced for a given degree of available tilt. Patients undergoing radiotherapy may be frail and their mobility may be limited, and reducing the ‘hop-on’ height of a subject support apparatus, i.e. the height of the subject support above the floor when in a neutral tilt position (e.g. when the subject support is parallel to the floor, or when the tilt angle is 0), is advantageous as it allows patients to more easily mount and position themselves on the subject support. Reducing the gap between the longitudinal sledge 216 and the subject support 212 is also advantageous for other reasons, for example because it reduces the chances that something may be caught in this gap, for example the fingers of a patient or clinician, as the subject support 212 tilts with respect to the longitudinal sledge 216.

As can be appreciated from the figures, the first guides 621, 622, 623, 624 are arranged to incline away from a centre of the plate 640. More specifically, the first guides 621, 622, 623, 624 are arranged to incline away from a centre line of the plate 640. In the arrangement depicted, the first guides 621, 622, 623, 624 incline away from a longitudinal centre line of the plate 640 and from the tilt axis 211. A first subset of the first guides, denoted by reference numerals 621 and 622, are positioned on a first side of the longitudinal sledge 216, and a second subset of the first guides, denoted by reference numerals 623 and 624, are positioned on an opposing, second side of the longitudinal sledge 216. The first and second subset of the first guides are thereby positioned either side of the tilt axis 211. The guides 621, 622 of the first subset of guides incline away from the centre of the plate and/or away from the tilt axis 211 in a first direction, and the guides 623, 624 of the second subset of guides incline away from the centre of the plate and/or away from the tilt axis 211 in a second direction, with the first direction being opposite the second direction. Each of the first subset of guides 621, 622 faces a respective one of the second subset of guides 623, 624. Accordingly, the subject support is biased to move towards a central neutral position, thus improving the stability of the subject support and guarding against large and sudden changes in the subject support tilt angle should there be a mechanical failure of any kind.

The subject support apparatus 114 further comprises first guide blocks 635, 636, 637, 638 disposed on the plate 640. In the arrangement depicted in the figures, the first guide blocks 635, 636, 637, 638 are disposed at the four corners of the plate 640. As with the arrangement described with respect to FIGS. 3 to 5, the first guide blocks 635, 636, 637, 638 are configured to slide along the inclined surface of the corresponding first guides 621, 622, 623, 624. The guides 621, 622, 623, 624 may comprise a plurality of guide rails in the manner described above, and the guide blocks 635, 636, 637, 638 may comprise a plurality of corresponding first carriages arranged to slide along the plurality of guide rails. The plurality of first carriages may be rotatably coupled to the plate 640 via a rotational coupling. For example, rotational coupling 639 depicted in FIG. 6c couples plate 640 to a carriage which forms part of guide block 637. These rotational couplings allow the plate 640 to tilt as the carriages move up and down the inclined surfaces of the guide rails.

As with other arrangements described herein, the apparatus comprises a tilting module positioned between the longitudinal sledge 216 and the subject support which comprises the plate 640 and a first actuator 625.

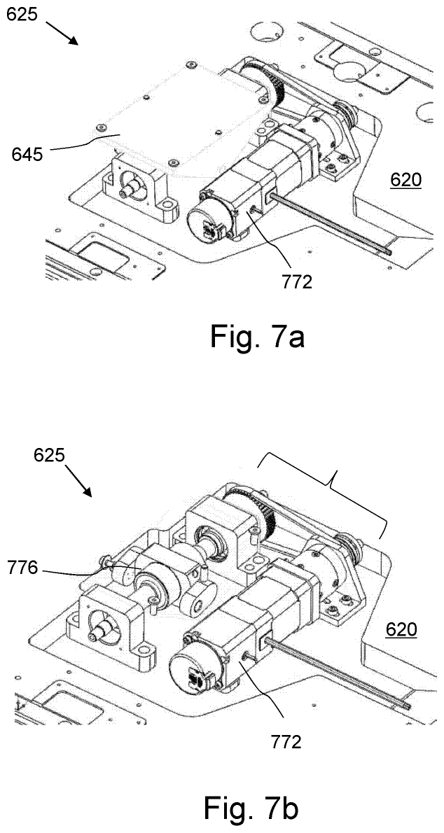

FIGS. 7a and 7b illustrate the first actuator 625. The plate 640 is coupled to an underside of the subject support such that the subject support tilts with the plate 640. The plate 640 is also coupled to the first actuator 625 such that movement generated by the first actuator 625 is translated into movement of the plate 640 along the first guides 621, 622, 623, 624, thereby causing the subject support to tilt relative to the plate 640.

The first actuator 625 is disposed on the longitudinal sledge 216. The first actuator is positioned in a recess of the longitudinal sledge 216, again so as to reduce the ‘hop-on height’, i.e. the gap between longitudinal sledge 216 and the support when the subject support apparatus 114 is in a neutral tilt position (e.g. when the subject support, or a top surface of the subject support, is parallel to the ground, or when the tilt angle is 0 relative to the horizontal). The first actuator 625 is a linear actuator and comprises an electric motor 772, a belt and pulley arrangement, and a lead screw or ball screw 776. The skilled person will appreciate that the first actuator 625 may take different forms in order to achieve the functionality described herein, and that rather than the form depicted in FIGS. 7a-7b the first actuator 625 may take any of a plurality of forms; for example, a simple piston as depicted in FIGS. 3-5.

The plate 640 is coupled to the first actuator 625 via a motion converter 645, which may be referred to as a motion converting unit or a motion converting element. The motion converter 645 may have a triangular prism shape. The motion converter 645 is rotationally coupled to the first actuator 625.

The motion converter 645 comprises axles or spindles which are configured to co-operate with apertures or indentations on the first actuator 625 in order to define a rotational coupling between the motion converter 645 and the linear actuator 625. It will be understood that the provision of spindles and corresponding indentations or apertures may be reversed. The motion converter 645 is rigidly coupled to the underside of the plate 640 and may form part of the plate 640.

In operation, when the first actuator 625 extends, the motion is passed via the motion converter 645 to the plate 640. Motion of the first actuator 645 causes movement of the motion converter 645, which in turn causes a tilting action of the plate 640, which in turn causes the subject support to tilt. Motion of the first actuator 625 along a movement axis is translated to, i.e. causes, movement of the plate 640 along the inclined first guides 621, 622, 623, 624, thereby causing the subject support to tilt about a first rotation axis relative to the base. In the arrangement depicted in FIGS. 6a-c, 7a and 7b, this rotation axis is substantially perpendicular to the movement axis of the first actuator 645.

By coupling the first actuator 625 to the plate 640, e.g. via the motion converter 645, force is applied directly to the tiltable plate. Because the tiltable plate is coupled directly to the tiltable subject support, an efficient transfer of energy and force is provided from the actuator, to the plate, and to the tiltable subject support. This is advantageous over a design which seeks to instead effect a tilting action by applying a force to a moveable inclined surface underneath the subject support in order to force the subject support upward, thereby causing a tilting action about a pivot point. Such a design would require the pivot point to be anchored between the base and upper surface, and such a design would create unnecessary strains and stresses at the pivot point and thus would be more susceptible to mechanical failure.

The subject support apparatus is depicted in the figures as having a tilt axis parallel to the longitudinal axis of the subject support. This may also be described as a roll rotation. However, it should be appreciated that the same mechanism may be used to provide a pitch rotation via reconfiguration and reorientation of the first guides. The subject support apparatus is depicted in the figures as being tiltable about only one axis of rotation, however it should be appreciated that a plurality of second inclined guides and second guide blocks may be provided, in the manner described herein, so as to allow the subject support of apparatus to tilt about a second tilt axis, such as a lateral or transverse axis.

### Movement of Subject Support

The tilting arrangement of the subject support apparatus 114 (e.g. 314), as described in FIGS. 6a-c, can allow for a tilt of the subject support of up to, for example, plus or minus 3 degrees from the horizontal on each longitudinal side of the subject support. A subject support apparatus may often comprise a cover or other component of a subject support receiving portion disposed generally beneath the longitudinal sides of subject support. Typically, this cover comprises a surface which runs substantially parallel to the subject support when the subject support is in a neutral (or ‘zero-tilt’) position (e.g. when the tilt angle is 0). Portions of the subject support which extend longitudinally along the side of the subject support and are disposed generally above such a cover may introduce a risk of trapping fingers, cables, tubes, and the like, when the subject support tilts. That is, the tilting of the subject support apparatus 114 may introduce a finger-trapping and/or cable-trapping risk when in such a configuration, as a limb, cable, tube or the like may become trapped between the extending portion and the substantially parallel cover as the subject support tilts. It is therefore beneficial to have an arrangement of the subject support and the subject support receiving portion, which may comprise a cover or the like, which is configured to alleviate such a risk. A number of such configurations are discussed below.

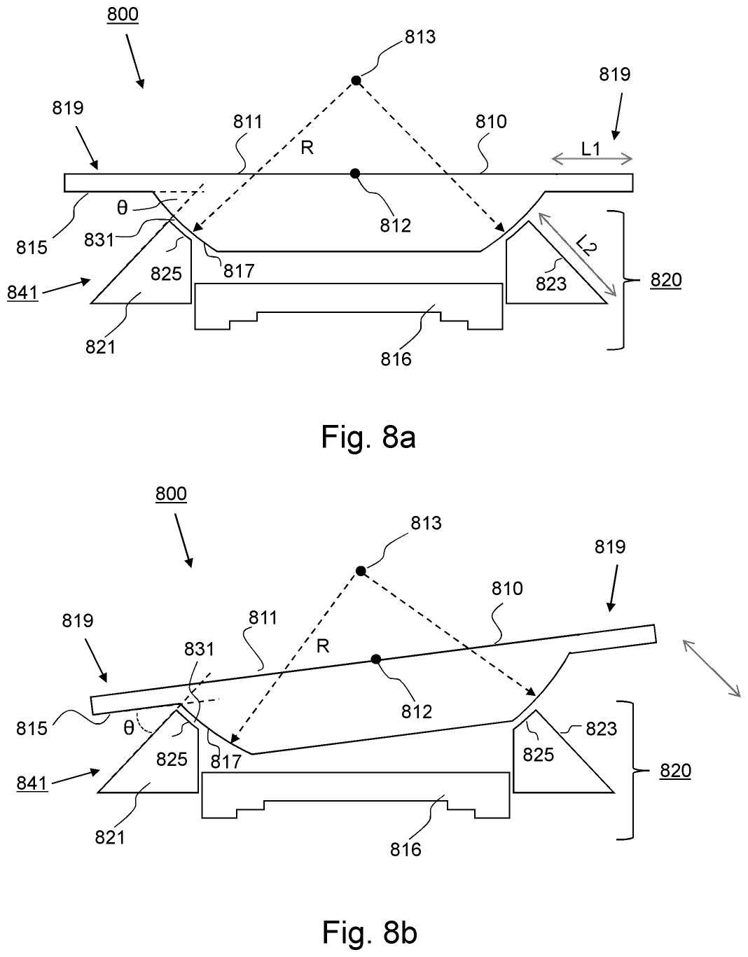

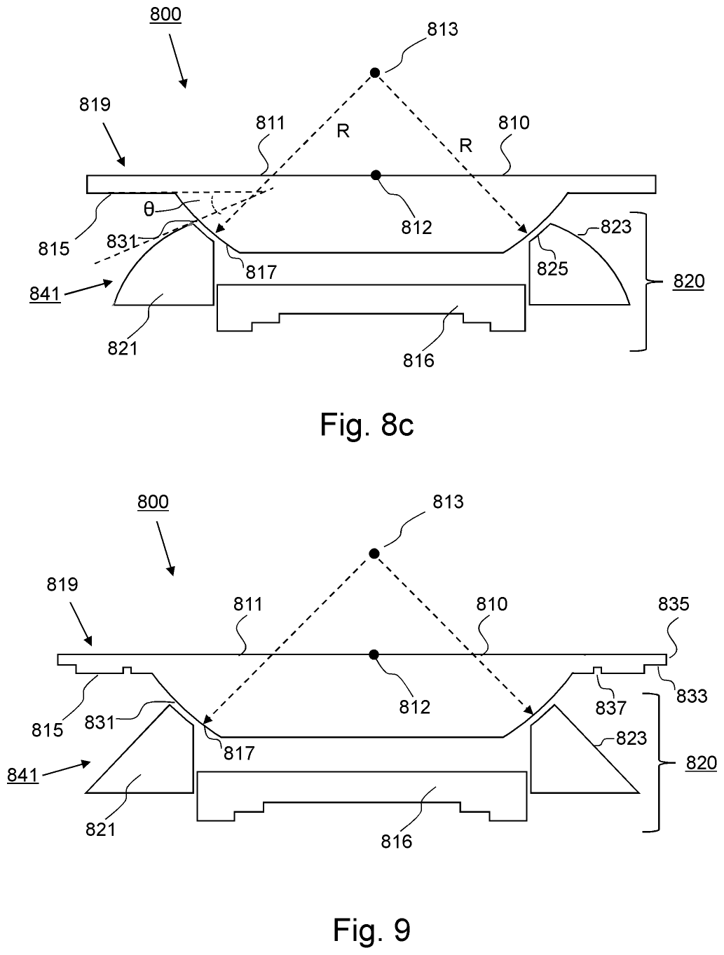

FIGS. 8a to 8c show a simplified section view of a subject support apparatus 800 for the radiotherapy device described above. Subject support apparatus 800 may be, for example, subject support apparatus 314. A number of elements of the subject support apparatus 800 have been omitted for simplicity. For the purposes of illustration, the description below and those illustrated in the accompanying figures are described as if both longitudinal sides of the apparatus comprise the disclosed features. In some embodiments, the described and illustrated features may indeed be present on both longitudinal sides of the subject support apparatus and/or the subject support receiving portion. However, in other embodiments, some or all of the features may only be present on one longitudinal side of the apparatus. For example, in some implementations, only one extending edge may be present. In other embodiments, the subject support apparatus may comprise an extending edge on each side.

The subject support apparatus 800 comprises a subject support receiving portion 820 and a subject support 810 which is tiltable relative to the subject support receiving portion 820 around a tilt axis 813. The tilt axis 813 may be an axis above a central longitudinal axis 812 of the subject support 810, that is, in line with a longitudinal axis passing through or along the centre of the subject support 810. In other words, the tilt axis 813 may be a longitudinal axis which is above the central longitudinal axis 812 of the subject support 810, e.g. along an imaginary line perpendicular to the horizontal axis extending from the central longitudinal axis 812 of the subject support 810.

The subject support receiving portion 820 supports the subject support 810. The subject support receiving portion 820 may comprise a platform, or may be comprised in the platform. That is, in some implementations, the platform comprises a subject support receiving portion, which may be a portion of the platform. In some implementations, the subject support receiving portion may be attached, e.g. removably attached, to the platform. It is to be understood that the subject support receiving portion may be incorporated by, attached to, part of, a component of, or coupled to a platform, or vice versa.

The subject support receiving portion 820 may further include side portions 821 and, in some embodiments, a longitudinal sledge 816. That is, the subject support receiving portion 820 may be generally disposed above the longitudinal sledge 816. In some implementations, intermediary components may be disposed between the subject support receiving portion 820, the subject support 810 and the longitudinal sledge 816. In some implementations, the subject support receiving portion 820 may comprise one or more of the side portions 821, which may be a cover or other removable or non-removable component configured to be coupled to the platform. The longitudinal sledge 816 includes similar or identical features to the longitudinal sledge 216 described above.

The subject support receiving portion 820 is disposed generally below the subject support 810, thereby providing support to an underside of the subject support 810. In some implementations, the subject support 810 may be disposed directly on the subject support receiving portion 820. In some implementations, the subject support 810 may be disposed on one or more intermediary components which are disposed on the subject support receiving portion 820.

The subject support apparatus 800 may further comprise a tilting module (not shown) which is configured to tilt the subject support 810 around the tilt axis 813.

### Subject Support

The subject support 810 is configured to support a patient, or subject. The subject support 810 may comprise at least a first longitudinal side wall 817 and a tabletop component. The tabletop component may comprise a first surface 811 and an extending edge 819 extending beyond the first longitudinal side wall 817. The extending edge may have an associated length L1, indicating the distance between where the first longitudinal side wall meets the extending edge and the end of the extending edge, measured distally. In some embodiments, the subject support 810 may further comprise a second longitudinal side wall and a second extending edge, extending beyond the second longitudinal side wall.

The first longitudinal side wall 817 of the subject support 810 is disposed generally below the first surface 811, and is dimensioned such that it can be received by the subject support receiving portion 820. As indicated above, the subject support receiving portion 820 may be a component of the platform, or a separate component which may be coupled to the platform 820. For example, the subject support receiving portion 820 may comprise a cover.

The first and second longitudinal side walls 817 may be curved, and may have a radius of curvature R. The first and second longitudinal side walls 817 may also be referred to as a curved portion throughout the description. In some implementations, a base of the subject support 810 may be curved such that, in a cross-sectional view of the subject support 810, the first and second longitudinal side walls 817 may form a circular arc having radius of curvature R. In other implementations, the subject support 810 may have a flattened portion between the first and second longitudinal side walls 817, shown in the figures as opposing the longitudinal sledge 816. It is to be understood that the radius of curvature need not be precisely circular. In other words, the first and/or second longitudinal side walls 817 may be substantially circularly curved. That is, the curvature of the first and/or second longitudinal side walls 817 may be slightly elliptical.

The subject support receiving portion 820 comprises a first longitudinal interior wall 825 and a first longitudinal exterior wall 823. The first longitudinal exterior wall 823 may be inclined at an angle relative to the horizontal axis. In some implementations, the first longitudinal exterior wall 823 may be straight, e.g. having a constant gradient. In some implementations, the first longitudinal exterior wall may be curved (e.g. may have a cross-section in the shape of a circular arc). In some implementations, the first longitudinal exterior wall 823 may have an associated length of L2, wherein the length L2 is greater than or equal to the length of the extending edge L1. The length L2 of the first longitudinal exterior wall may include only an inclined and/or curved surface. In some implementations, the first longitudinal exterior wall 823 may be formed by multiple components. In such cases, the length L2 may be a total length of these multiple components. For example, a first component may sloped or curved, as illustrated in the figures, for a length L2a, and a second component may have a different slope with a length L2b, or have a substantially vertical slope.

The extending edge 819 and the first longitudinal exterior wall 823 may form an open angle, when the subject support 810 is in any tilt configuration. That is, the distance between a bottom surface, e.g. underside, of the extending edge 819 and the first longitudinal exterior wall 823 may increase distally. In other words, the distance between the bottom surface of the extending edge 819 and the first longitudinal exterior wall 823 at a first point along the extending edge may be less than the distance between the bottom surface of the extending edge 819 and the first longitudinal exterior wall 823 at a second point along the extending edge 819, the second point being closer to the end of the extending edge 819 than the first point, and the first point being closer to the central longitudinal axis of the subject support 810 than the second point.

In some implementations, the subject support receiving portion 820 may further comprise a second longitudinal interior wall 825 and a second longitudinal exterior wall 823, which may be arranged similarly to the first longitudinal interior wall 825 and the first longitudinal exterior wall 823, respectively, for example on an opposite side of the subject support 810.

In some implementations, the first longitudinal side wall 817 of the subject support has a curvature such that each point along the side surface is displaced from the central longitudinal axis of the subject support 810 by a constant distance R. This value may be referred to as the radius of curvature. Tilting of the subject support causes the first longitudinal side wall 817 to emerge from or retract within the platform. In some implementations, a portion of the first longitudinal side wall 817 may protrude from the subject support receiving portion 820 in all tilt configurations. That is, a portion of the first longitudinal side wall 817 may remain outside of the subject support receiving unit 820 in all tilt configurations. It is to be understood that the radius of curvature need not be precisely circular. In other words, the first longitudinal side wall 817 may be substantially circularly curved. That is, the curvature of the first and/or second longitudinal side walls 817 may be slightly elliptical.

The extending edge 819 of the subject support may have a smaller thickness than the rest of the subject support, and may be formed as a lip along one or both longitudinal sides of the subject support 810. The extending edge 819 comprises an upper surface, which may form part of the first surface 811, and a lower surface 815 on the underside of the extending edge 819.

In some implementations, the extending edge(s) 819 is planar with the tabletop component of the subject support. Alternatively, one or more extending edge may be inclined relative to the plane of the surface of the tabletop component. That is, the extending edge 819 may be angled relative to the surface of the tabletop component.

FIG. 8a illustrates the subject support 810 in a neutral tilt position. In the neutral tilt position, the tilt angle may be zero or substantially zero. In other words, the patient positioning surface may be parallel, or substantially parallel, to the ground or floor.

FIG. 8b illustrates the subject support 810 in a maximally tilted position. The subject support is said to be at a maximally tilted position when the subject support is tilted through a maximum tilt angle to either one side or the other. In some implementations, the subject support may be tilted between +/−3° from the horizontal. At the maximally tilted position of the subject support, a distance between the lower surface of the extending edge 819 and the first longitudinal exterior wall 823 increases with increasing displacement from the central longitudinal axis 812. In particular, this distance may be the distance between the lower surface of the extending edge 819 which is below the central longitudinal axis 812, e.g. tilted down, as shown in the figure, and the first longitudinal exterior wall 823.

At the maximally tilted position, the extending edge is angled at an angle, marked θ. This angle may be referred to as a minimum angular difference, relative to the first longitudinal exterior wall 823 of the subject support receiving portion 820. The angle θ may be the angle between the extending edge and the first longitudinal exterior wall 823 of the subject support receiving portion 820 when the subject support is in a maximally tilted position, and in particular in a tilt direction in which the extending edge approaches the first longitudinal exterior wall 823, e.g. when the extending edge is below the horizontal axis, e.g. below the position of the extending edge when the subject support 810 is in a neutral tilt position, as illustrated in the figure.

The minimum angular difference may therefore to the difference between:
a) the angle of inclination of the first longitudinal exterior wall 823 of the subject support receiving portion 820 relative to the horizontal axis, and
b) the angle of the extending edge 819 relative to the horizontal axis when the subject support is in the maximally tilted position

The angle of inclination of the first longitudinal exterior wall 823 of the subject support receiving portion may be selected such that the minimum angular difference is greater than a threshold angular difference. That is, the subject support receiving portion 820, e.g. the first longitudinal exterior wall thereof, may be arranged such that the minimum angular difference exceeds the threshold angular difference. In implementations in which the extending edge 819 is planar with the surface of the tabletop component, the angle of the extending edge 819 relative to the horizontal axis when the subject support is in the maximally tilted position may be the tilt angle. The threshold angular difference may be based on a number of factors, such as material, coefficient of friction, size and the like. In some implementations, the threshold angular difference may be approximately 30°, and preferably 36°. The minimum angular difference may be therefore at least the threshold angular difference, and preferably greater than the threshold angular difference. A larger minimum angular difference is desirable, as a larger angle may create a larger region between the extending edge and the subject support receiving portion 820.

FIG. 8c illustrates a subject support receiving portion in which the first longitudinal exterior wall is curved. That is, in some implementations, the first longitudinal exterior wall of the subject support receiving portion 820 is curved with a radius of curvature denoted r(ext). In such implementations, the angle of inclination of the first longitudinal exterior wall 823 of the subject support receiving portion may be an angle of a plane tangent to the curve of the first longitudinal exterior wall 823 at a point along the curve at which the tangential slope is at a minimum, or at a point within 2-3 mm of the first longitudinal side wall 817. For example, a tangent to the curved first longitudinal exterior wall 823 as shown in FIG. 8c is at a minimum at the flattest point.

FIG. 9 depicts a cross sectional view of a subject support apparatus 800 for the radiotherapy device. In some embodiments, the extending edge 819 may have a cutout region, or a recess 833. The recess 833 may be arranged for receiving at least part of an accessory attached to the subject support. For example, the recess 833 may be used to clamp or otherwise couple a cable or other component to the subject support. Providing a recess 833 for receiving at least part of an accessory prevents things, such as patient's fingers or clinician's fingers, cables and tubes, from being trapped when the subject support rotates. In some implementations, the recess 833 may comprise an accessory surface located on a side of the subject support which is opposite to the surface of the tabletop component. The recess 833 may be defined by a region of reduced thickness, referred to and depicted as an end portion 835 of the extending edge 819.

The recess 833 may, in some implementations, extend along the full length of the extending edge, e.g. in a longitudinal direction, or along a portion of the length of the extending edge. For example, there may be one or more regions of the extending edge with said recess, allowing an accessory to be attached to the extending edge at one or more regions along the length of the subject support.

In some implementations, the first longitudinal side wall 817 may comprise a portion which does not oppose the first longitudinal interior wall 825 of the subject support receiving portion 820 as the subject support 810 is tilted around the tilt axis 813. This portion of the first longitudinal side wall 817 may remain exposed during any degree of tilt of the subject support 810 about the tilt axis 813. The degree of tilt of the subject support 810 about the tilt axis 813 may be limited by this portion, e.g. the subject support 810 may have a maximum angle of tilt at the tilt angle where only this portion is exposed above of the first longitudinal side wall 817 is exposed above the subject receiving portion 820. In some implementations, a second longitudinal side wall 817 may comprise a corresponding portion which does not oppose the second longitudinal interior wall 825 of the subject support receiving portion 820. In some implementations, the first longitudinal interior wall 825 may be short, for example 1-2 cm in length.

The subject support receiving portion 820 supports the subject support 810. The subject support receiving portion 820 may comprise a platform, or may be comprised in the platform. That is, in some implementations, the platform comprises a subject support receiving portion, which may be a portion of the platform. In some implementations, the subject support receiving portion may be attached, e.g. removably attached, to the platform. It is to be understood that the subject support receiving portion may be incorporated by, attached to, part of, a component of, or coupled to a platform, or vice versa.

The subject support receiving portion 820 may further include side portions 821 and, in some embodiments, a longitudinal sledge 816. That is, the subject support receiving portion 820 may be generally disposed above the longitudinal sledge 816. In some implementations, intermediary components may be disposed between the subject support receiving portion 820, the subject support 810 and the longitudinal sledge 816. In some implementations, the subject support receiving portion 820 may comprise one or more of the side portions 821, which may be a cover or other removable or non-removable component configured to be coupled to the platform. The longitudinal sledge 816 includes similar or identical features to the longitudinal sledge 216 described above.

The subject support receiving portion 820 further comprises an opening for receiving the subject support 810, and may be configured such that a gap 831 exists along at least one longitudinal side of the subject support 810 where the subject support 810 is received by the platform 820. The gap 831 may be small enough to prevent finger trapping, and may be less than 8 mm. Further, according to the implementations described herein, the gap 831 may remain substantially constant as the subject support 810 is tilted about the tilt axis 813.

In some implementations, the first longitudinal side wall 817 and the second longitudinal side wall 817 of the subject support are curved, having a radius of curvature R defined with respect to tilt axis 813. The tilt axis, as described above, may be in line with the central longitudinal axis of the subject support. In some implementations, an underside of the subject support may comprise the first longitudinal side wall 817 and the second longitudinal side wall 817. In some implementations, the underside of the subject support may further comprise a bottom surface, which may be substantially flat or curved. In some implementations, the radius of curvature of the underside is constant, such that the profile of the underside of the subject support corresponds to an arc of a circle.

The side portions are dimensioned such that the gap 831 exists between the first longitudinal side wall 817 of the subject support 810 and the side portions 821 of the subject support receiving portion 820. The gap 831 may be bounded by at least part of the first longitudinal interior wall 825 and second longitudinal interior wall 825 of the subject support receiving portion 820. The side portions 821 may be, for example, a cover for part of the apparatus, and may be used to cover, for example, cables connecting the subject support 810 to the subject support receiving portion 820, e.g. the platform. The first longitudinal exterior wall 823 and second longitudinal exterior wall 823 are defined on the outward-facing surfaces of the side portions.

That is, the first and second longitudinal interior walls 825 of the subject support receiving portion 820 may each comprise a curved region configured to receive the subject support. The curved region has a radius of curvature greater than the radius of curvature of the first and second longitudinal side walls of the subject support. The difference in radii of curvature enables a gap 831 to be formed between the first and second longitudinal side walls 817 of the subject support 810, and the subject support receiving portion 820. Said gap remains constant as the subject support is tilted.

As shown in FIGS. 8a to 9, on each longitudinal side of the subject support apparatus 800 a region 841 between the subject support 810 and the subject support receiving portion 820 is formed, wherein boundaries of the region 841 are defined by the first longitudinal exterior wall 823 of the subject support receiving portion, the lower surface 815 of the extending edge 819 and the first longitudinal side wall 817 of the subject support.

This region forms due to the extending edge 819 of the subject support 810 extending over the first longitudinal exterior wall 823. When the subject support is in a neutral tilt position (e.g. a tilt angle of) 0°, a segment of the first longitudinal side wall 817, also referred to as the curved portion, further bounds the region 841 as described above.

Angle θ may defined where an extension of the lower surface 815 of the extending edge 819 towards a perpendicular plane through the central longitudinal axis 812 and an extension of the first longitudinal exterior wall 823 of the side portion towards a perpendicular plane through the central longitudinal axis 812 intersect. A first angle can be defined as the angle of inclination of the first longitudinal exterior wall relative to a horizontal axis, and a second angle can be defined as the angle of the extending edge 819 relative to the horizontal axis when the subject support 810 is in a maximally tilted position, as described above. The horizontal axis may be defined as a lateral axis across the top surface of the tabletop component when in a neutral position. The angle θ can be defined as the difference between the first angle and second angle. The angle θ decreases as the subject support tilts, such that the displacement between opposing faces of the first longitudinal exterior wall 823 and the lower surface 815 of the extending edge B819 decreases. Thus, the segment of the first longitudinal side wall 817 which bounds the region 841 defined above decreases as θ decreases.

In some implementations, a maximum angle of tilt of the subject support may be achieved when the lower surface 815 of the extending edge 819 is in contact with the first longitudinal exterior wall 823 at its innermost point. As a result of this maximum tilt, the region 841 may be bounded only by the lower surface 815 of the extending edge 819 and the first longitudinal exterior wall 823.

As the subject support tilts, e.g. during treatment or imaging, there is a risk that a limb may become trapped in the region between an extending edge 819 of the subject support 810, and the subject support receiving portion 820 supporting the subject support 810. In the implementations described herein, the subject support 810 is configured such that the region 841 does not pose a finger trapping risk for any tilt configuration of the subject support 810. This is due to the inclination of the first longitudinal exterior wall, which causes a limb placed in the region 841 to be pushed outward from the central longitudinal axis as the tilt of the subject support 810 increases. As the subject support 810 tilts, the extending edge 819 presses downwards onto a limb in region 841, and the limb comes in contact with the first longitudinal exterior wall 823 and is thus pushed outward, e.g. in a direction away (e.g. distally) from the central longitudinal axis 812 due to the inclination of the first longitudinal exterior wall 823.

As described above, the threshold angular difference between the first angle of the first longitudinal exterior wall 823 and the second angle of the extending edge 819 is determined such as to prevent the trapping of a limb in the region 841. The determination of the threshold angular difference may involve determining the minimum size required for the region 841 to prevent a limb being trapped within it. In some implementations, the minimum angular threshold may be predetermined as 30 degrees, and preferably 36 degrees. In some implementations, where the first longitudinal exterior wall comprises a curved surface, this minimum angle will increase with increasing distance from the tilt axis. Variations in the minimum angular threshold may be possible, and such variations may be dependent upon the material (and associated friction) of the extending edge and/or the first longitudinal exterior wall. Thus, the limb cannot become trapped in the region between the extending edge 819 of the subject support and the platform structure 820. For example, if the first longitudinal exterior wall 823 has a low coefficient of friction, such as a painted surface, a polished surface, carbon fibre, plastic or the like, then the minimum angular threshold may be smaller. If the longitudinal exterior wall 823 has a high coefficient of friction, such as a rough surface, the minimum angular threshold may be larger.

The relationship between the length L1 of the extending edge 819 and the length L2 of the first longitudinal exterior wall 823 allows an open angle to be present between the extending edge 819 and the first longitudinal exterior wall 823, e.g. the displacement between the extending edge 819 and the first longitudinal exterior wall 823 increases in the outward direction (e.g. distally), e.g. from a central longitudinal axis 812 of the subject support 810. This ensures that a limb in contact with either the extending edge 819 or the first longitudinal exterior wall 823 can be easily retracted from the region 841, thus alleviating the risk of finger-trapping.

The incline or curve of the first longitudinal exterior wall 823 may provide a further advantage in that liquid leaking from the upper surface 811 of the subject support tabletop component may be directed away from the subject support receiving portion 820 of the apparatus due to the incline or curve of the first longitudinal exterior wall 823. This feature can thus prevent the ingress of liquid into the apparatus.

FIG. 9 depicts a cross sectional view of a subject support apparatus 800 for the radiotherapy device described above, with the addition of several optional features. A number of elements of the subject support apparatus have been omitted for simplicity. For the purposes of illustration, the below embodiments are described as if both longitudinal sides of the apparatus comprise the disclosed features. However, as described above, in other embodiments, some or all of the features may only be present on one longitudinal side of the apparatus.

As shown in FIG. 9, a liquid directing feature or groove 837 is located on the lower surface of the extending edge 819 along one or both longitudinal side(s) of the subject support 810. That is, the groove 837 may be provided on one side, e.g. on an extending edge extending beyond the first longitudinal side wall 817 of the subject support, or on both sides, e.g. on the extending edges 819 extending beyond the first and second respective longitudinal side walls 817 of the subject support. The groove 837 may be small in diameter, e.g. 1-2 mm and extend along the longitudinal side of the subject support 810, and may be configured such that its presence minimises fluid ingress into the underside of the subject support, and/or into the subject support receiving portion 820. The groove 837 may be further configured such that it exhibits capillary action in order to direct fluid away from the underside of the subject support. The groove 837 may run substantially parallel to the central longitudinal axis 812.

The groove 837 may be further configured to direct fluid away from the gap 831 between the subject support receiving portion 820 and the first longitudinal side wall 817 of the subject support 810.

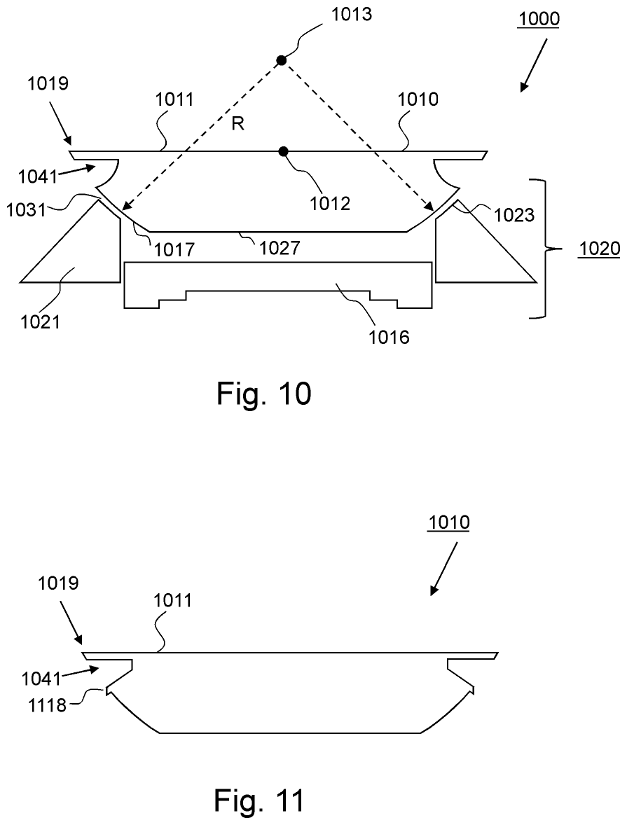

FIG. 10 shows a simplified section view of a subject support apparatus 1000 for the claimed radiotherapy apparatus. A number of elements of the subject support apparatus have been omitted for simplicity. For the purposes of illustration, as described above, the below embodiments are described as if both longitudinal sides of the apparatus comprise the disclosed features. However, in other embodiments, some or all of the features may only be present on one longitudinal side of the apparatus.

The subject support apparatus 1000 comprises a subject support receiving portion 1020 and a subject support 1010, which is tiltable relative to the subject support receiving portion 1020 around a tilt axis 1013. Similarly to the subject support receiving portion 820 described above with reference to FIGS. 8 and 9, the subject support receiving portion 1020 may be a component or region of the platform, or an additional component arranged on the platform to receive the subject support 1010. The subject support receiving portion 1020 may comprise a platform, and these two terms may be used interchangeably. It is to be understood that the subject support receiving portion may be incorporated by, (e.g. removably) attached to, part of, a component of, or coupled to a platform, or vice versa. The subject support receiving portion 1020 may support the subject support 1010. The subject support receiving portion 1020 may includes two side portions 1021 which may be, for example, covers. The subject support receiving portion 1020 may, in some embodiments, comprise a longitudinal sledge 1016, or maybe coupled to a component which comprises a longitudinal sledge 1016. These features may have equivalent or similar properties to the corresponding features of the subject support apparatus 800 described above.

The subject support receiving portion 1020 comprises a first longitudinal interior wall 1023 and, in some implementations, a second longitudinal interior wall 1023. The first and second longitudinal interior walls may be curved, having a radius of curvature R2 (not shown).

The subject support 1010 is configured to support a patient, or subject, on a first surface 1011 (also referred to as a patient positioning surface or subject support surface). The subject support 1010 comprises a first longitudinal side wall 1017 and a second longitudinal side wall 1017, which are curved with a radius of curvature R.

The radius of curvature R is less than the radius of curvature R2 of the first and second longitudinal interior walls 1023 of the subject support receiving portion. In some implementations, the radius of curvature R may be less than the radius of curvature R2 by some threshold distance, e.g. between 4 and 10 mm, and preferably 8 mm.

The surface of the subject support 1010 facing the platform or the subject support receiving portion 1020 may be referred to as the underside of the subject support 1010. The underside may comprise the first and second longitudinal side walls 1017. In some implementations, the underside may have a constant radius of curvature, such that its cross-section forms an arc of a circle of radius R. In some implementations, such as that depicted in FIG. 10, the underside may further comprise a flattened portion 1027 between the first longitudinal side wall 1017 and the second longitudinal side wall 1017. Said flattened portion 1027 may be substantially parallel to the ground when the subject support is in a neutral tilt position. In some implementations, each point on the first and second longitudinal side walls 1017 is an equal distance (R) from the tilt axis 1013.

In some implementations, an optional cut-away region 1041 along each longitudinal side of the first longitudinal side wall 1017 may be provided to create edge portions of reduced thickness 1019 which extend outwardly over the cut away region 1041. As the first and second longitudinal side walls 1017 of the subject support have a profile of substantially circular arcs, the distal points of the edge portions remain at most the constant distance R from the central longitudinal axis. That is, the maximum width of the subject support is double the radius of curvature R.

The cut-away region 1041 may create a space for a limb to interact with the apparatus, without the risk of trapping the limb in the region between the subject support receiving unit 1021 and the first and/or second longitudinal side wall 1017 of the subject support. A gap arises between the first longitudinal interior wall 1023 of the subject support receiving portion 1020 and the first longitudinal side wall 1017 of the subject support 1010 as a consequence of the radius of curvature of the first longitudinal side wall 1017 being smaller than the radius of curvature of the first longitudinal interior wall 1023. The gap 1031 located in the region where the subject support 1010 is received by the subject support receiving portion 1020 will remain constant, which further reduces the risk of trapping. For example, such a gap 1031 may exist between the side portion 1021, which may be a cover, and the first longitudinal side wall 1017.

FIG. 11 depicts a simplified section view of an embodiment of a subject support 1010 for the radiotherapy apparatus. Features of the subject support apparatus may have equivalent or similar properties to the corresponding features of the subject support apparatus 1000 described above.

The subject support 1010 is configured in a substantially similar manner to that depicted in FIG. 10, and comprises a cut-away region 1041 along one or both longitudinal side(s) wall of the subject support 1010. The cut-away region 1041 results in the creation of an edge portion 1019 of reduced diameter which extends outwardly. That is, the maximum width of the subject support remains double the radius of curvature of the first and second longitudinal side walls. In some implementations, the lower surface of the cut away region 1041 is angled downwards and, optionally, further comprises a lip 1118 at its distal end which extends outwardly.

The protruding lip 1118 may prevent fluid ingress into the region below the subject support 1010 by directing fluid away from a gap 1031 between the subject support 1010 and a subject support receiving portion or platform (e.g. subject support receiving portion 1020 shown in FIG. 10) disposed generally below the subject support 1010.

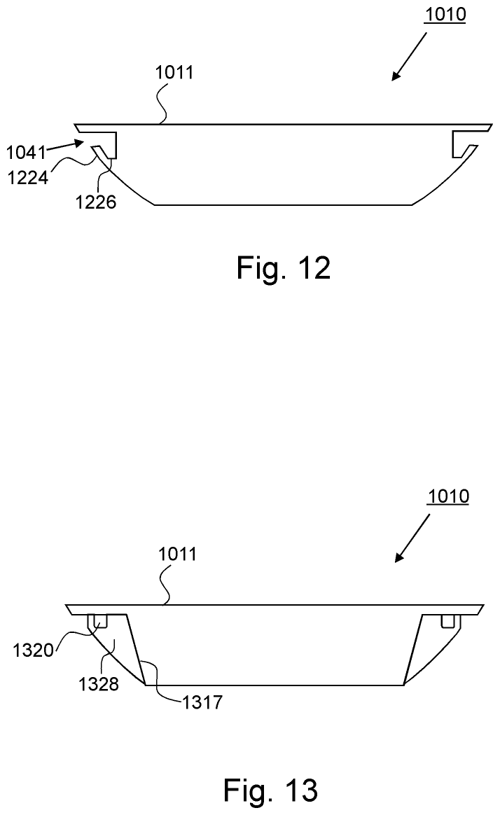

FIG. 12 depicts a simplified section view of an embodiment of a subject support 1010 for the radiotherapy apparatus. Features of the subject support 1010 may have equivalent or similar properties to the corresponding features of the subject support apparatus 1000 described above.

The subject support 1010 is configured in a substantially similar manner to that depicted in FIG. 10, and comprises a cut-away region 1041 along one or both longitudinal side walls 1017 of the subject support 1010. The cut-away region 1041 results in the creation of an edge portion 1019 of reduced diameter which extends outwardly. That is, the maximum width of the subject support remains double the radius of curvature of the first and second longitudinal side walls. A lower surface 1226 of the cut-away region 1041 comprises a lip 1224 which extends along the longitudinal side of the subject support 1010. The lip 1224 protrudes in an angled upward direction, towards the distal end of an underside surface of the edge portion 1019. The angled lip is dimensioned such that its maximal distance from the central longitudinal axis of the subject support is the radius of curvature R. The lip 1224 may form a groove defined by lower surface 1226, which may be arranged to exhibit capillary action. Such an arrangement would prevent liquid from accumulating in or on the subject support receiving portion, and from entering the apparatus.

The configuration of the cut-away region 1041 may prevent fluid ingress into the region below the subject support 1010, as fluid will be channeled through the cut-away region along the longitudinal side of the apparatus, and will exit the apparatus upon reaching the end of the cut-away region.

FIG. 13 depicts a simplified section view of an embodiment of a subject support 1010 for the radiotherapy apparatus. Features of the subject support apparatus 1010 may have equivalent or similar properties to the corresponding features of the subject support apparatus 1000 described above.

The subject support 1010 is configured in a substantially similar manner to that depicted in FIG. 10, and comprises cut-away region 1041 along one or both longitudinal side walls of the subject support 1010. The cut-away region results in the creation of an edge portion 1019 of reduced diameter which extends outwardly. That is, the maximum width of the subject support remains double the radius of curvature of the first and second longitudinal side walls. In this implementation, the cut-away region 1041 is configured to accommodate an insert 1328. That is, the subject support receiving portion may further comprise the insert 1328. The insert 1328 extends longitudinally along the cut-away region, and attaches to a side surface 1317 of the subject support 1010 and an underside surface of the edge portion 1019. The insert 1328 comprises a cavity 1320 which extends longitudinally along the insert 1328.

The configuration of the insert 1328 and the associated cavity 1320 may prevent fluid ingress into the region below the subject support 1010, as fluid will be channeled through the cavity 1320 along the longitudinal side of the apparatus, and will exit the apparatus upon reaching an opening at the end of the cavity 1320.

The insert 1328 may be detachable in order to accommodate cleaning of the apparatus.

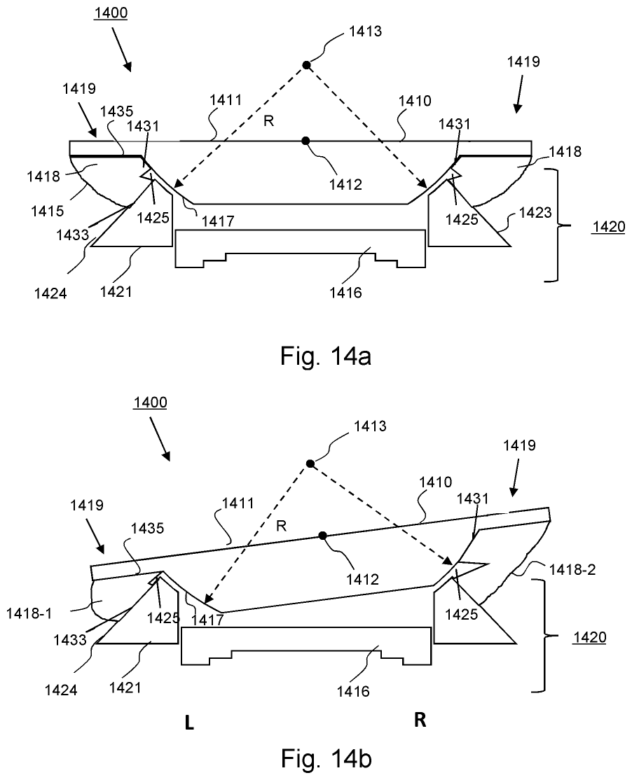

FIGS. 14a and 14b show a simplified section view of a subject support apparatus 1400 for the radiotherapy device described above. Subject support apparatus 1400 may be, for example, subject support apparatus 314. A number of elements of the subject support apparatus 1400 have been omitted for simplicity. For the purposes of illustration, the description below and those illustrated in the accompanying figures are described as if both longitudinal sides of the apparatus comprise the disclosed features. In some embodiments, the described and illustrated features may indeed be present on both longitudinal sides of the subject support apparatus and/or the subject support receiving portion. However, in other embodiments, some or all of the features may only be present on one longitudinal side of the apparatus.

The subject support apparatus 1400 comprises a subject support receiving portion 1420 and a subject support 1410 which is configured to tilt relative to the subject support receiving portion 1420. The subject support 1410 may be tiltable around a tilt axis 1413. The tilt axis 1413 may be an axis above a central longitudinal axis 1412 of the subject support 1410, that is, in line with a longitudinal axis passing through or along the centre of subject support 1410. In other words, the tilt axis 1413 may be a longitudinal axis which is above the central longitudinal axis 1412 of the subject support 1410, e.g. along an imaginary line perpendicular to the horizontal axis extending from the central longitudinal axis 1412 of the subject support 1410.

The subject support receiving portion 1420 is configured to support the subject support 1410. The subject support receiving portion 1420 may comprise a platform, or may be comprised in a platform. That is, in some implementations, the platform comprises a subject support receiving portion, which may be a portion of the platform. In some implementations, the subject support receiving portion may be attached, e.g. removably attached, to the platform. It is to be understood that the subject support receiving portion may be incorporated by, attached to, part of, a component of, or coupled to a platform, or vice versa.

The subject support receiving portion 1420 may comprise features which have similar or identical properties to the corresponding features of any other subject support apparatus described herein. In some embodiments, the subject support receiving portion 1420 may comprise side portions 1421, and/or a longitudinal sledge 1416. In some implementations, intermediary components may be disposed between the subject support receiving portion 1420 and the longitudinal sledge 1416. In some implementations, the subject support receiving portions may comprise one or more of the side portions 1421, which may be a cover or other removable or non-removable component configured to be coupled to the platform. The longitudinal sledge 1416 includes similar or identical features to the longitudinal sledge 216 described above.

The subject support receiving portion 1420 is disposed generally below the subject support 1410, thereby providing support to an underside of the subject support 1410. In some implementations, the subject support 1410 may be disposed directly on the subject support receiving portion 1420. In some implementations, the subject support 1410 may be disposed on one or more intermediary components which are disposed on the subject support receiving portion 1410.

The subject support apparatus may further comprise a tilting module (not shown) which is configured to tilt the subject support 1410 relative to the subject support receiving portion 1420. The tilting module may be configured to tilt the subject support relative to the tilt axis 1413.

The subject support 1410 may comprise features which have similar or identical properties to the corresponding features of any other subject support described herein. For example, the subject support apparatus may comprise features which have similar or identical properties to the corresponding features of subject support 810.

The subject support 1410 is configured to support a patient, or subject. The subject support 1410 may comprise at least a longitudinal side wall 1417 and a tabletop component. The tabletop component may comprise a surface 1411, e.g. a subject support surface, and an extending edge 1419 extending beyond the longitudinal side wall 1417. In some implementations, the subject support 1410 may further comprise a second longitudinal side wall and a second extending edge, extending beyond the second longitudinal side wall.

The longitudinal side wall 1417 of the subject support 1410 is disposed generally below the surface 1411, and is dimensioned such that it can be received by the subject support receiving portion 1420.

As indicated above, the subject support receiving portion 1420 may be a component of the platform, or a separate component which may be coupled to the platform. For example, the subject support receiving portion 1420 may comprise a cover. In some implementations, the longitudinal side wall 1417 and a second longitudinal side wall 1417 may comprise features which have similar or identical properties to the corresponding features of subject support 810.

For example, in some embodiments, the first and second longitudinal side walls 1417 may be at least partially curved, and may have a radius of curvature R, as described in FIGS. 8 to 13. The first and second longitudinal side walls 1417 may also be referred to as a curved portion throughout the description. In some implementations, a base of the subject support 1410 may be curved, or partially curved, such that, in a cross-sectional view of the subject support 1410, the first and second longitudinal side walls 1417 may form a circular arc having radius of curvature R. In other implementations, the subject support 1410 may have a flattened portion between the first and second longitudinal side walls 1417, shown in the figures as opposing the longitudinal sledge 1416. It is to be understood that the radius of curvature need not be precisely circular. In other words, the first and/or second longitudinal side walls 1417 may be substantially circularly curved. That is, the curvature of the first and/or second longitudinal side walls 1417 may be slightly elliptical. Although FIGS. 14a and 14b depict curved longitudinal side walls 1417, this is merely exemplary.

The subject support receiving portion 1420 comprises a longitudinal exterior wall 1424. In some embodiments, the longitudinal exterior wall 1424 may be inclined at an angle relative to the horizontal axis. In some implementations, the longitudinal exterior wall 1424 may be straight (e.g. may have a constant gradient). In other implementations, the longitudinal exterior wall 1424 may be curved (e.g. may have a cross section in the shape of a circular arc). In some implementations, the longitudinal exterior wall 1424 may be formed by multiple components. For example, a first component may be sloped or curved, and a second component may have a different slope, or a substantially vertical slope.

In some embodiments, the extending edge 1419 and the longitudinal exterior wall 1424 may form an open angle when the subject support 1410 is in any tilt configuration, as described with reference to FIG. 8.

In some implementations, the longitudinal side wall 1417 of the subject support has a curvature such that each point along the side surface is displaced from the central longitudinal axis of the subject support 1410 by a constant distance R. This value may be referred to as a radius of curvature. Tilting of the subject support causes the longitudinal side wall 1417 to emerge from or retract within the platform. In some implementations, a portion of the longitudinal side wall 1417 may protrude from the subject support receiving portion 1420 in all tilt configurations. That is, a portion of the longitudinal side wall 1417 may remain outside of the subject support receiving unit 1420 in all tilt configurations. It is to be understood that the radius of curvature need not be precisely circular. In other words, the longitudinal side wall 1417 may be substantially circularly curved. That is, the curvature of the longitudinal side wall 1417 may be slightly elliptical.

In some implementations, the longitudinal side wall 1417 may be straight (e.g. may be sloped, or have a constant gradient). In some implementations, the longitudinal wall 1417 may be formed by multiple components. For example, a first component may be sloped or curved, and a second component may have a different slope, or a substantially vertical slope.

In some implementations, the subject support receiving portion 1420 may further comprise a second longitudinal exterior wall 1424, which may be arranged similarly to the longitudinal exterior wall 1424, respectively, for example on an opposite side of the subject support 1410.

The extending edge 1419 of the subject support may have a smaller thickness than the rest of the subject support, and may be formed as a lip along one or both longitudinal sides of the subject support 1410. The extending edge 1419 comprises an upper surface, which may form part of the first surface 1411, and a lower surface on the underside of the extending edge 1419.

In some implementations, the extending edge(s) 1419 is planar with a tabletop component of the subject support. Alternatively, one or more extending edge may be inclined relative to the plane of the surface of the tabletop component. That is, the extending edge 1419 may be angled relative to the surface of the tabletop component.

Subject support apparatus 1400 further comprises a deformable guide 1418. The deformable guide 1418 may be disposed generally below the extending edge 1419 of the subject support. In embodiments of the apparatus which do not comprise the optional extending edge 1419, the first deformable guide may be disposed generally above a side portion 1421 of the subject support receiving portion 1420.

The deformable guide 1418 may extend in a longitudinal direction, and in some implementations, may extend along the full length of the subject support 1410. In other implementations, the deformable guide may extend partially along the length of the subject support 1410 in a longitudinal direction. In some implementations, the deformable guide may comprise a plurality of sections which extend along the length of the subject support 1410 intermittently, or at regular intervals. In other words, the deformable guide may extend along at least one section of the length of the subject support 1410.

The deformable guide 1418 comprises a first face 1431 and a second face 1433. The first face is arranged to be in contact, at least in part, with the longitudinal side wall 1417 of the subject support 1410. The second face 1433 is arranged to be in contact, at least in part with the subject support receiving portion 1420, e.g. the longitudinal exterior wall 1424 of the subject support receiving portion 1420. In some implementations, the first face 1431 may be in contact, at least in part, with the subject support 1410, e.g. the longitudinal side wall 1417 of the subject support 1410, and the second face 1433 may be in contact, at least in part with the longitudinal exterior wall 1424 of the subject support receiving portion 1420. In other words, in some implementations the first face 1431 and the second face 1433 may be in contact, at least in part, with the longitudinal side wall 1417 of the subject support 1410 and the longitudinal exterior wall 1424 of the subject support receiving portion 1420 respectively, throughout a range of tilt configurations of the subject support apparatus 1400. Being in contact may comprise being removably attached, non-removably attached, or merely touching. In some embodiments, the respective faces of the deformable guide 1418 may be attached (either removably or non-removably) to at least one of the subject support 1410 and the subject support receiving portion 1420 through mechanical and/or chemical means (e.g. adhesive).

In some embodiments, the length of the portion, or part, of the first interior face 1431 which is arranged to be in contact with the longitudinal side wall 1417 of the subject support 1410 may correspond to a tilt angle of the subject support 1410. This may be a first tilted position of the subject support, and may correspond to a maximally tilted position of the subject support. The maximally tilted position of the subject support may be, for example, a tilt position at which no further tilt is possible, or a tilt position which is determined (e.g. by a setting or by an operator) to be a maximal tilt position although no physical limitations are met. In other words, the length of the portion, or part, of the first face 1431 which is arranged to be in contact with the longitudinal side wall 1417 may be such that it is not received into the subject support receiving portion 1420 during any tilt of the subject support 1410 relative to the tilt axis. In particular, in some embodiments, the length of the portion, or part, of the first face 1431 which is arranged to be in contact with the longitudinal side wall 1417 may be such that it will not be received into the subject support receiving portion 1420 at a maximum tilt angle of the subject support 1410. This allows for the tilt of the subject support 1410 relative to the tilt axis 1413, without causing damage to (e.g. by tearing) the deformable guide 1418.

In some implementations, the length of the portion, or part, of the first face 1431 which is arranged to be in contact with the longitudinal side wall 1417 of the subject support 1410 may be less than or equal to 15 mm.

In some implementations, the length of the portion, or part, of the first face 1431 which is arranged to be in contact with the longitudinal side wall 1417 may be permanently or removably attached to the longitudinal side wall 1417. The second face may be permanently or removably attached to the longitudinal exterior wall 1424 of the subject support receiving portion 1420. This may involve chemical means of attachment, such as adhesives or gluing, or mechanical means of attachment, such as a locking mechanism, e.g. a tongue and groove construction, and/or using Velcro tape, e.g. Velcro tape attached to the longitudinal exterior wall 1424 and to the deformable guide (e.g. the Velcro tape may be attached to an upper face 1435 of the deformable guide 1418). The Velcro tape may be attached to the longitudinal exterior wall 1424 and/or the deformable guide by an adhesive such as 3M adhesive. Further examples include a hook, which may be glued or seamed onto the deformable guide 1418. Such mechanisms may enable the deformable guide 1418 to be easily attachable and removable, for example for sterilisation, cleaning and/or replacement. In implementations where one or both of the attachments are removable, the configuration of the apparatus can allow for the removal, replacement and sterilisation of the deformable guide 1418 between radiotherapy treatment sessions.

Other implementations may allow for a greater or lesser length of the portion, or part, of the first face 1431 to be in contact with the longitudinal side wall 1417 of the subject support 1410. In such implementations, the length of the portion, or part, of the first face 1431 which is in contact with the longitudinal side wall may depend on a tilt angle of the subject support 1410 relative to the tilt axis 1413. For example, a subject support apparatus which has a greater angle of tilt (e.g. a greater maximum angle of tilt) may require a lesser length of the portion, or part, of the first face to be in contact with the longitudinal side wall, and vice versa.

The first face 1431 may further comprise a first recess 1425. The first recess 1425 may be a cut-out or recessed portion of the first face, the cut-out or recessed portion extending in the longitudinal direction. The first face 1431 may be configured such that the first recess 1425 is positioned on a lower portion of the first face 1431. The dimensions of the first recess 1425 may vary as the subject support 1410 tilts about the tilt axis 1413, as is explained in more detail further below. The first recess 1425 can reduce or prevent tearing of the deformable guide 1418 due to the tilting of the subject support 1410.

A deformation in the deformable guide 1418 may result in a corresponding deformation of the first recess 1425. An expansion of the deformable guide 1418 may lead to an expansion of the first recess 1425, e.g. may allow or cause the first recess 1425 to expand. A compression or contraction of the deformable guide may lead to a compression or contraction of the first recess 1425. In other words, the size of the first internal recess 1425 may depend on the relative expansion or contraction of the deformable guide 1425. This allows for the subject support 1425 to tilt relative to the tilt axis, without causing damage to the deformable guide 1418. In other words, the deformation of the internal recess aids the deformation of the deformable guide 1418 as the subject support 1410 tilts relative to the tilt axis 1413.

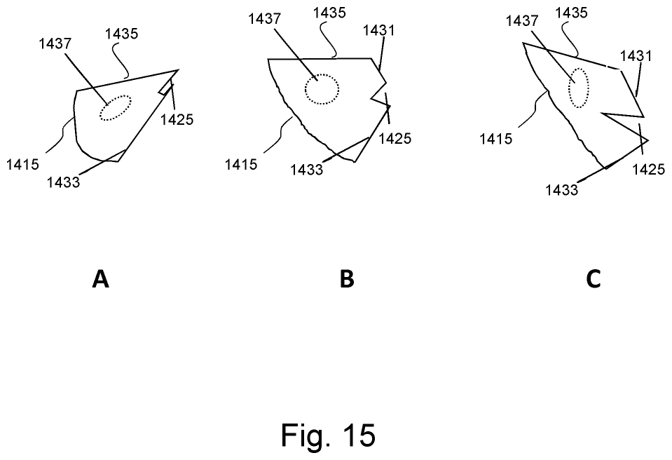

In some implementations, the deformable guide 1418 may comprise a channel 1437 which extends longitudinally through the deformable guide 1418. This channel may also be referred to as an internal recessed portion 1437 of the deformable guide 1418 (as shown in FIG. 15). Such a channel may be enclosed entirely within the deformable guide, and may extend longitudinally along the deformable guide. That is, the longitudinal ends of the channel may be closed or sealed by the deformable guide. In other implementations, the channel may have at least one open end, in which at least one longitudinal end of the channel is open, e.g. not covered by material of the deformable guide. In some implementations, both ends of the channel may be open, such that the channel extends longitudinally through the whole length of the deformable guide. In other words, the deformable guide may be hollow, and/or may comprise a longitudinal hole extending through the guide. Such a feature allows for the deformable guide 1418 to undergo increased deformation. For example, in implementations where the deformable guide 1418 is a compressible guide, the presence of a hollow cavity or longitudinal hole through the guide 1418 allows for increased compression distance. The use of a hollow and/or channel 1437 may also enable more durable materials to be used.

In some implementations, the first face 1431 of the deformable guide 1418 may be fixed to the longitudinal side wall 1417 of the subject support 1410. In other words, the first face 1431 of the deformable guide 1431 may be permanently or removably attached to longitudinal side wall 1417 of the subject support. This may involve chemical means of attachment, such as adhesives or gluing, or mechanical means of attachment, such as a locking mechanism, e.g. a tongue and groove configuration, a slit, a slot and/or using Velcro tape. For example, Velcro tape may be attached to the longitudinal exterior wall 1424 and to the deformable guide (e.g. the Velcro tape may be attached to an upper face 1435 of the deformable guide 1418). The Velcro tape may be attached to the longitudinal exterior wall 1424 and/or the deformable guide by an adhesive such as 3M adhesive. Further examples include a hook, which may be glued or seamed onto the deformable guide 1418. Such mechanisms may enable the deformable guide 1418 to be easily attachable and removable, for example for sterilisation, cleaning and/or replacement. In some implementations, the first face 1431 of the deformable guide 1431 is adhered, at least in part, to the longitudinal side wall 1417 of the subject support 1410 using, for example, glue.

The deformable guide 1418 is composed of a deformable material, such as a resiliently and/or elastically deformable material. In some implementations, the deformable guide 1418 may be an elastically deformable guide. In such implementations, the deformable guide 1418 is fully or substantially elastically deformable. The deformable guide 1418 may be a resiliently deformable guide. In other words, the deformable guide 1418 may be able to withstand an elastic deformation of the guide without deforming plastically. That is, the deformable guide 1418 may not be permanently deformed as a result of a deformation. For example, upon release of a force deforming (e.g. compressing) the deformable guide 1418, the deformable guide 1418 may return to its original shape.

In some implementations, the deformable guide 1418 may comprise an expanded plastic. For example, the deformable guide 1418 may comprise a foam plastic such as foamed neoprene. In some implementations, the deformable guide may comprise a material of a specified density, which may allow for friction with other components of the apparatus, or wear due to contact with the other components of the apparatus, to be reduced.

Further, the deformable guide may comprise a coating, such as a low friction coating. Such a coating may be a coating which reduces friction and/or wear on the deformable guide. The low friction coating may be, for example, a Teflon coating or film, or a polyethylene coating or film.

The deformable guide 1418 may further comprise an exterior face 1415. The exterior face 1415 may comprise a surface which is not in contact with the subject support 1410 or the subject support receiving portion 1420, e.g. the longitudinal side wall 1417 of the subject support 1410, or the longitudinal exterior wall 1424 of the subject support receiving portion 1420. The exterior face 1415 of the deformable guide 1418 may comprise an exterior surface of the deformable guide which is most distal to the tilt axis 1413. In some implementations, the exterior face 1415 may be straight (e.g. may be vertical or substantially vertical, or may have a constant gradient) when the subject support 1410 is in a neutral tilt position. In other implementations, the exterior face 1415 may be curved (e.g. may have a cross section in the shape of a circular arc) when the subject support 1410 is in a neutral tilt position. In such implementations, the exterior face may, at the neutral tilt position, have a radius of curvature defined with respect to the tilt axis. In implementations wherein the curve of the longitudinal side wall 1417 has a radius of curvature defined with respect to the tilt axis 1413, the radius of curvature of the exterior face of the deformable guide 1418 may be greater than the radius of curvature of the longitudinal side wall 1417. In other words, the exterior face 1415 of the deformable guide may be shaped such that it protrudes in an outward direction from the subject support apparatus 1400.

In some implementations, the deformable guide 1418 may further comprise an upper face 1435. The upper face 1435 can be the uppermost surface of the deformable guide 1418, and may be substantially parallel to the subject support surface 1411. In some implementations, where the subject support surface 1411 comprises an extending edge 1419, the upper face 1435 may be in contact, at least in part, with the underside of the extending edge 1419. In other implementations, the upper face 1435 may be an exposed face of the deformable guide 1418.

The upper face 1435 may be permanently or removably attached to the underside of the extending edge 1419. This may involve chemical means of attachment, such as adhesives or gluing, or mechanical means of attachment, such as a mechanical locking mechanism such as a tongue and groove construction, a slit, a slot and/or using Velcro tape. For example, Velcro tape may be attached to the longitudinal exterior wall 1424 and to the deformable guide (e.g. the Velcro tape may be attached to an upper face 1435 of the deformable guide 1418). The Velcro tape may be attached to the longitudinal exterior wall 1424 and/or the deformable guide by an adhesive such as 3M adhesive. Further examples include a hook, which may be glued or seamed onto the deformable guide 1418. Such mechanisms may enable the deformable guide 1418 to be easily attachable and removable, for example for sterilisation, cleaning and/or replacement. In some implementations, the upper face 1435 of the deformable guide 1418 is adhered, at least in part, to the underside of the extending edge 1419 of the subject support 1417 using, for example, glue.

In some implementations, the subject support apparatus 1400 may further comprise a further deformable guide, which may be arranged similarly to the deformable guide 1418, respectively, for example on an opposite side of the subject support 1410. In such implementations, the deformable guide 1418 is referred to as a first deformable guide 1418, and the further deformable guide is referred to as a second deformable guide 1418. The first deformable guide 1418 may comprise features as described in any implementation disclosed herein. The second deformable guide 1418 can comprise features with similar or identical properties to the features of the first deformable guide 1418. In some implementations, the first deformable guide 1418 and the second deformable guide 1418 may be identical. In other implementations, the second deformable guide 1418 may comprise additional or different features relative to the first deformable guide 1418. For example, the first deformable guide 1418 may comprise channel 1437, and the second deformable guide may omit this feature. Any such combinations of features may be used.

FIG. 14a illustrates the subject support 1410 in a neutral tilt position, where the tilt angle may be zero or substantially zero. In other words, the patient positioning surface may be parallel, or substantially parallel, to the horizontal (e.g. the ground or floor).

In the neutral tilt position, the deformable guide 1418 may be in a neutral state or a state of deformation. This may be a state of partial deformation. In some implementations, this may correspond to a state of partial compression and/or contraction. That is, the first deformable guide may be partially compressed when the subject support 1410 is in the neutral tilt position.

In implementations which comprise a further or additional deformable guide 1418, described herein as a second deformable guide 1418, the second deformable guide 1418 may also be in a neutral deformation state or in a state of partial deformation. In some implementations, this may correspond to a state of partial compression. That is to say, the second deformable guide may be partially compressed when the subject support 1410 is in the neutral tilt position. The first and second deformable guides may be in the same or a substantially equivalent state of compression in the neutral tilt position. In implementations where the first and second deformable guides 1418 are composed of the same or similar materials, the first and second deformable guides are in the same or a similar state of compression in the neutral tilt position.

FIG. 14b illustrates the subject support 1410 in a first tilted position. Such a first tilted position may be, for example, a maximally tilted position of the subject support. The subject support is said to be at a tilted position when the subject support is tilted through a tilt angle to either one side or the other. In some implementations, the subject support may be tilted between +/−3° from the horizontal. At a first tilted position of the subject support, for example −3° from the horizontal, a distance between a lower surface of the extending edge 1419 and the longitudinal exterior wall 1424 of the subject support receiving portion may be at a minimum. In particular, this distance may be the distance between the lower surface of the extending edge 1419 which is below the central longitudinal axis 1412, e.g. tilted down, and the longitudinal exterior wall 1424. At a second tilted position, for example +3° from the horizontal, a distance between the lower surface of the extending edge 1419 and the longitudinal exterior wall 1424 of the subject support receiving portion may be at a maximum. Said maximum may be, for example, the result of a physical limitation or a setting which has been determined, for example by a user, or as indicated by a treatment plan. In particular, this distance may be the distance between the lower surface of the extending edge 1419 which is above the central longitudinal axis 1412, e.g. tilted up, and the longitudinal exterior wall 1424. The first and second tilted positions are illustrated in FIG. 14b, and denoted as L and R respectively.

As the skilled person will appreciate, and as illustrated in FIG. 14b, one longitudinal side of the apparatus being in the first tilted position will cause an opposing longitudinal side of the apparatus to be in the second tilted position, and vice versa.

As the subject support 1410 tilts relative to the tilt axis 1413, the deformable guide 1418 may be in a state of increasing deformation. In other words, as the subject support tilts, the deformable guide may be in a state of relative contraction or expansion, depending on the direction of the tilt. The deformation of the deformable guide 1418 increases as an angle between the subject support 1410 and the longitudinal exterior wall 1424 of the subject support receiving portion 1420 decreases. This may be an angle between an extending edge 1419 of the subject support surface 1411 and the longitudinal exterior wall 1424 of the subject support receiving portion. That is, as the angle between a bottom surface, e.g. underside, of the extending edge 1419 and the longitudinal exterior wall 1424 decreases, the deformation of the deformable guide may increase. In implementations where the deformable guide 1418 comprises a compressible guide, the compression of the deformable guide 1418 increases as the angle between an extending edge 1419, or an underside thereof, of the subject support surface 1411 and the longitudinal exterior wall 1424 of the subject support receiving portion increases.

As the subject support 1410 approaches the first tilted position of the subject support 1410, the deformable guide 1418-1 deforms. That is, as the subject support 1410 tilts towards the first tilted position, the deformation of the deformable guide increases. In implementations where the deformable guide is a compressible guide, the deformable guide 1418-1 is increasingly compressed as the subject support 1410 approaches the first tilted position.

In implementations where the deformable guide 1418 comprises an internal recess 1425, the internal recess 1425 may deform in a substantially proportional manner to the deformation of the deformable guide 1418. That is, as the tilting of the subject support 1410 approaches the first tilted position, the internal recess 1425 may compress in a substantially proportional manner to the deformation of the deformable guide 1418-1.

The deformation of the deformable guide 1418-1 may cause the deformable guide 1418-1 to protrude and/or expand outwardly from the apparatus. As the subject support 1410 tilts towards, or approaches, the first tilted position, the increasing deformation of the deformable guide 1418-1 may cause the deformable guide 1418-1 to protrude outwardly, e.g. in a direction away from the subject support 1410. That is, decreasing the angle between the subject support 1410 and the longitudinal exterior wall 1424 of the subject support receiving portion 1420 may cause the deformable guide 1418 to protrude or expand outwardly from the subject support apparatus. In other words, a distance from the tilt axis 1413 to a most distal point on the deformable guide 1418-1 may increase with increasing deformation of the deformable guide 1418-1. Said outward expansion of the deformable guide 1418-1 may push any fingers or limbs out of the way, preventing finger trapping.

In some implementations, the exterior face 1415 of the deformable guide 1418-1 may comprise the most distal point on the deformable guide 1418-1 relative to the tilt axis 1413. In such implementations, the deformation of the deformable guide 1418-1 may cause the exterior face 1415 of the deformable guide 1418-1 to protrude outwardly from the apparatus. In other words, a distance from the tilt axis 1413 to the first exterior face 1415 of the deformable guide 1418-1 increases with increasing deformation of the deformable guide 1418-1. That is, the tilting of the subject support 1410 towards the first tilted position can cause the deformable guide to compress or contract in a substantially downward direction, relative to the horizontal axis, and expand in an outward direction relative to the subject support 1410.

As such, any limb, body part or cabling which may be in contact with the extending edge 1419, the deformable guide 1418-1, or the first exterior wall 1424 of the subject support receiving portion 1420 will be pushed in an outward direction as the subject support 1410 tilts towards the first tilted position, alleviating the risk of finger-trapping.

At the first tilted position of the subject support 1410, the deformable guide 1418-1 may be in a state of increased deformation. This may be a state of maximum deformation. In implementations where the deformable guide 1418-1 is a compressible guide, the deformable guide 1418-1 may be in a state of increased compression, or in a state of maximum compression in this first tilted position. The internal recess 1425, if present, is in a state of increased or maximum compression. In some implementations, in the first tilted position of the subject support 1140 the internal recess 1425 is compressed such that the recess is negligible.

As the subject support 1410 approaches the second tilted position of the subject support 1410, the deformable guide 1418-2 deforms. That is, as the subject support 1410 tilts towards the second tilted position, the deformation of the deformable guide increases. In implementations where the deformable guide is a compressible guide, the deformable guide 1418-2 expands as the subject support 1410 approaches the second tilted position.

In implementations where the deformable guide 1418 comprises an internal recess 1425, the internal recess 1425 may deform in a substantially proportional manner to the deformation of the deformable guide 1418. That is, in the second tilted position of the deformable guide 1418-2, the internal recess 1425 may expand in a substantially proportional manner to the deformation of the deformable guide 1418-2.

In some implementations, the exterior face 1415 of the deformable guide 1418-2 may comprise the most distal point on the deformable guide 1418-2 relative to the tilt axis 1413. In such implementations, the deformation of the deformable guide 1418-2 may cause the exterior face 1415 of the deformable guide 1418-1 to expand or stretch. In other words, a distance from the tilt axis 1413 to the exterior face 1415 of the deformable guide 1418-1 decreases with increasing deformation of the deformable guide 1418-1. In other words, the tilting of the subject support 1410 as it approaches the second tilted position causes the deformable guide to expand upwards, relative to the horizontal axis, and the upward expansion of the deformable guide 1418-2 may cause the exterior face 1415 of the deformable guide 1418-2 to move inwards, or towards the subject support 1410.

At the second tilted position of the subject support 1410, the deformable guide 1418-2 is in a state of increased deformation. This may be a state of maximum deformation. As indicated previously, the state of maximum deformation may be a state of deformation beyond which no further deformation is possible, or a state of deformation at which a maximum tilt angle is reached, for example in accordance with a setting (e.g. from a user and/or a treatment plan). In implementations where the deformable guide 1418-2 is a compressible guide, the deformable guide 1418-1 may be in a state of decreased or minimal compression in this second tilted position. This state of minimal compression may also be known as a state of expansion, which, in some implementations, may be a ‘full-release’ or neutral state.

In some implementations, the subject support apparatus 1400 may further comprise a further deformable guide 1418, positioned on the opposite side of the apparatus. In describing such implementations, reference is made to the respective deformable guides as the first deformable guide 1418, and the second deformable guide 1418. The second deformable guide 1418 may comprise features with similar or identical properties to the features of the first deformable guide 1418. In some implementations, the first deformable guide 1418 and the second deformable guide 1418 may be identical. In other implementations, the second deformable guide 1418 may comprise additional or different features relative to the first deformable guide 1418.

It will be appreciated that one longitudinal side of the apparatus being in the first tilted position will cause an opposing longitudinal side of the apparatus to be in the second tilted position, and vice versa. Thus, where the first deformable guide 1418 is in a state of increased contraction or compression, the second deformable guide will be in a state of decreased contraction or compression, and vice versa. The relative deformation of the first deformable guide and the second deformable guide may be substantially the same when the subject support 1410 is in the neutral tilt position, as shown in FIG. 14a.

FIG. 15 depicts a cross-section view of the deformable guide 1418 at various tilt positions of the subject support apparatus 1400. The configuration of the deformable guide 1418 in a first tilted position is denoted A on the figure, the configuration of the deformable guide 1418 in the neutral tilt position is denoted B on the figure, and the configuration of the deformable guide 1418 in the second tilted position, denoted C on the figure.

Subject support apparatus 1400 allows for the alleviation of finger-trapping risks due to the tilting of the subject support 1410 relative to the subject support receiving portion 1420 before or during treatment. The presence of the first deformable guide can prevent a patient from placing a limb or finger in the region in which there is a finger-trapping risk, for example underneath the extending edge 1419 of the apparatus. In particular, in cases where a comatose, anaesthetised or unconscious patient is receiving treatment, the apparatus 1400 provides a means to alleviate the risk of trapping a limb or finger of such a patient in the finger trapping region. Such patients are not capable of retracting a limb or a finger from a finger-trapping region themselves, thus the apparatus provides a means by which the offending finger or limb is pushed away from the finger-trapping region as the apparatus tilts, without requiring any effort from the patient.

In addition, the subject support apparatus 1400 provides for the alleviation of finger-trapping risks without any impact on the design of the subject support 1410. The subject support apparatus 1400 can be compatible with a number of existing subject support designs, and can be compatible with a number of existing subject support receiving portion designs. That is, the finger-trapping solution offered by the subject support apparatus 1400 can be fitted to existing designs without any impact on the design of the existing apparatus. This finger-trapping solution can also be fitted to other subject support apparatus embodiments described herein, without any impact on the design of the relevant subject support apparatus.

Another advantage offered by the subject support apparatus 1400 is that there is no impact on the minimum table loading height where this design is implemented. That is, the presence of the deformable guide or guides between the subject support and the subject support receiving portion does not affect the minimum table loading height of the subject support. The deformable guide or guides can be dimensioned so as to fit neatly between the existing subject support and the subject support receiving portion. This can involve fitting one or more deformable guides underneath an extending edge of the subject support, or in the absence of an extending edge this can involve fitting the deformable guide or guides such that the deformable guide or guides do not extend above the subject support. The one or more deformable guides may be easily installed, removed and replaced as necessary.

As such, the subject support apparatus 1400 provides a low cost and easy to implement apparatus for the alleviation of finger-trapping risks.

### Cable Routing

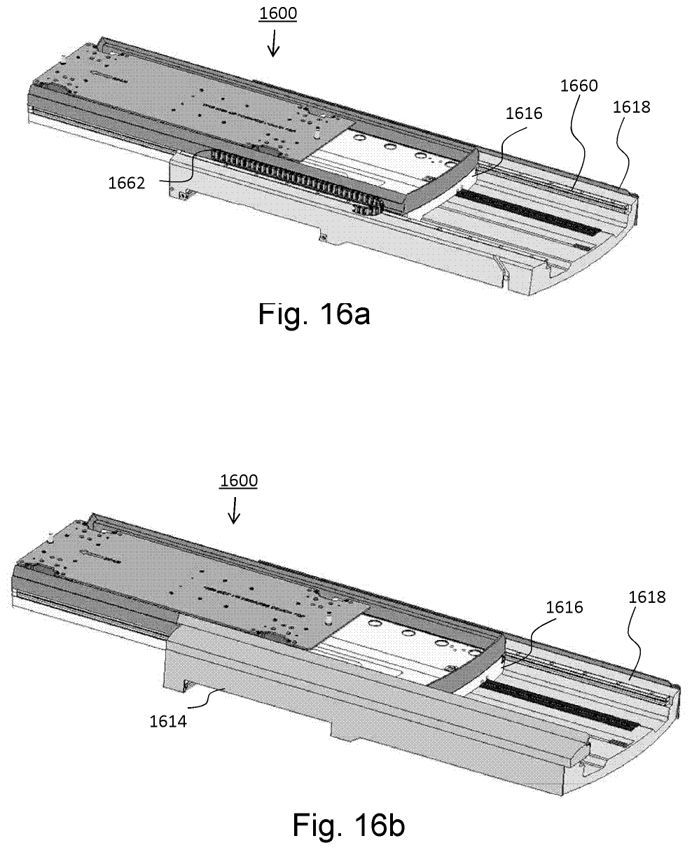

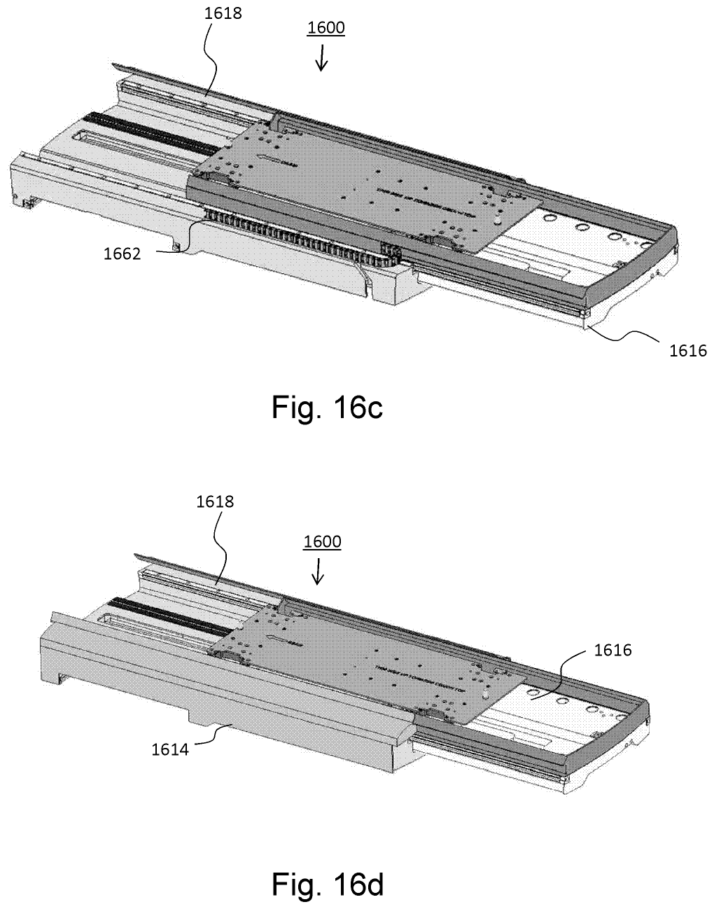

FIGS. 16a-d illustrate an aspect of a subject support apparatus 1600, in which some details have been omitted for simplicity.

FIGS. 16a-d depict a longitudinal sledge 1616 movably coupled to a platform 1618 disposed generally underneath the longitudinal sledge. Such a longitudinal sledge 1616 can be used to support the subject support. The platform 1618 may comprise a recessed portion into which the longitudinal sledge is received. The recessed portion can comprise grooves which complement the shape of an underside of the longitudinal sledge such as to allow the smooth movement of the longitudinal sledge 1616 along the platform 1618. In some embodiments, the longitudinal sledge 1616 may translate along guide rails 1660 attached to the platform 1618. The guide rails 1660 may be located within the recessed portion of the platform, or along the associated grooves.

A movable connection (not shown) between the subject support and the platform 1618 is provided. This movable connection can be an interface board which is movable or translatable relative to the platform 1618. The interface board electrically connects the subject support with at least one electrical cable in order to electrically connect the subject support with another component of the apparatus via the electrical cable. The interface board may be physically coupled, e.g. attached, to the subject support. For example, such a cable may electrically connect the subject support to an external power supply, or to the platform itself. One or more cables may be routed along the platform 1618, or routed within the platform 1618, in order to connect with, for example, components of the radiotherapy apparatus. Such electrical connections may include, for example, a connection to a tilting module of the subject support, wherein the tilting module is configured to control a tilt of the subject support relative to the platform.

Utilising an interface board to electrically connect the subject support with at least one electrical cable in order to electrically connect the subject support with another, e.g. at least one, component of the apparatus via the electrical cable eliminates or reduces a finger trapping risk. Typically, an apparatus of this type may comprise a number of individually connected electrical cables which connect the subject support to other components of the apparatus. Should such a subject support tilt or move relative to a platform disposed generally below, individual cables may move along an open groove, become slack or taut, or become generally displaced such that they introduce a finger trapping risk. Providing a movable connection or interface board through which all cables electrically connect to the subject support allow for the cables to be routed in such a manner as to avoid moving along an open groove, or to avoid become generally displaced. Cables may also be routed to avoid regions of the apparatus which pose known finger trapping risks.

In some embodiments, a cable chain 1662 may be provided. Such a cable chain can provide a cable management function, as the electrical cable or cables which electrically connect the subject support to other components of the radiotherapy apparatus may be maintained within the cable chain. The cable chain 1662 can be positioned on its side, such that the height of the cable chain can be kept the same as the roll mechanism. As such, the cable chain 1662 does not increase the patient step-on height of the table.

As shown in FIG. 16b, the apparatus may further comprise a cover 1614 which covers at least part of a side portion of the platform 1618. Such a cover 1614 may be removably attachable to the platform 1618. In addition, the cable chain 1662, along with the associated cables contained within the cable chain, are maintained within the cover 1614. This further reduces the risk of finger trapping or cable damage.

In order to arrange, maintain or clean underneath or around the cables, the cover 1614 can be detached from the platform 1618, facilitating easy access to the cable assembly.

FIGS. 16a-b, and FIGS. 16c-d depict the movement of the subject support and longitudinal sledge 1616 along the platform 1618. It can be seen that the subject support and the sledge 1616 have translated along the platform 1618. Further, the cable chain 1662 can be seen to move with the subject support, such as to maintain the electrical connection between the subject support and any external cables. Such an arrangement allows for cables to be neatly maintained within the cable chain 1662 for all subject support configurations.

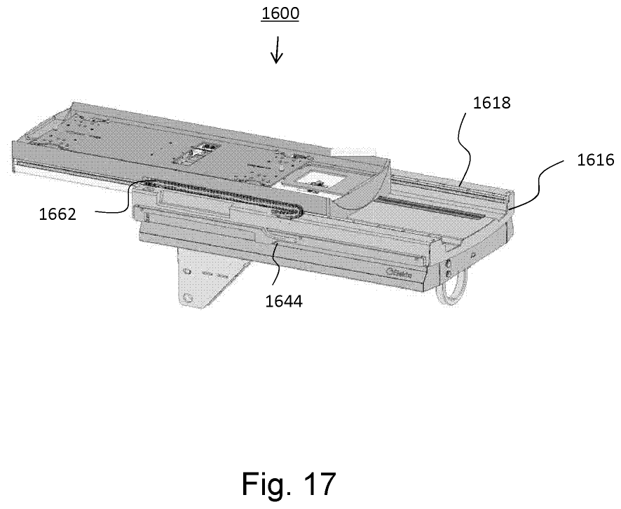

FIG. 17 depicts an embodiment of the apparatus of FIGS. 16a-c, wherein the electrical connection provided between the subject support and one or more external cables comprises at least one cable which is routed within the platform 1616 to provide an electrical connection to part of a lower lateral sledge 1644.

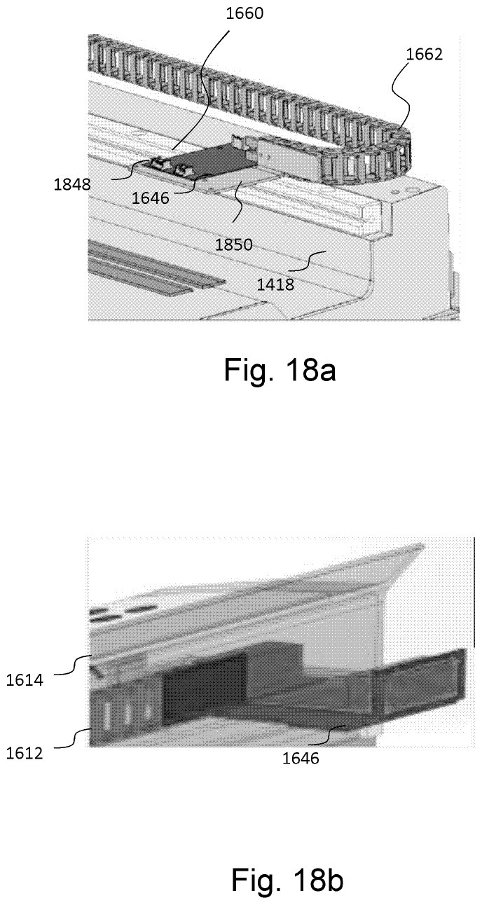

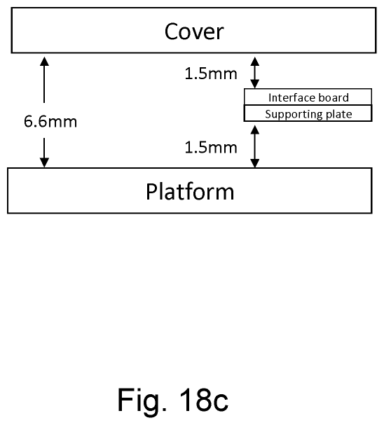

FIGS. 18a-c depict a detailed view of the movable connection or interface board between the longitudinal sledge 1616 (not shown) and the platform 1618 of the subject support apparatus 1600.

In FIG. 18a, the longitudinal sledge 1616 and cover 1614 have been removed from the Figure to expose the interface board 1646. It can be seen that the interface board may be partially or fully supported by the platform 1618, the platform 1618 being disposed generally below the interface board 1646. The subject support apparatus 1600 may comprise a slot configured to receive the interface board. Such a slot may be defined by a gap between the cover (not shown) and the platform 1618.

The interface board may be translatable relative to the platform, and may translate along a slot, e.g. a groove or a gap, defined between the platform 1618 and the cover (not shown). Such a slot or gap may be configured to accommodate the movement of the interface board 1646 relative to the platform 1618.

A movement of the subject support relative to the platform 1618, as shown in FIGS. 16a-d, may cause a movement of the interface board 1646. The interface board 1646 can be attached to the longitudinal sledge 1616 and/or the subject support (both not shown), via a cable or otherwise, such that a movement of the subject support relative to the platform 1618 causes a corresponding movement of the interface board 1646. For example, such movements may be translational movements, wherein as the longitudinal sledge translates along the guide rails 1660, the interface board 1646 undergoes a corresponding translational movement.

The movement of the longitudinal sledge 1616 relative to the platform 1618 may expose part of the slot or gap defined in the apparatus.

Further, the interface board 1646 may be mounted to the subject support or the longitudinal sledge 1616, such that the interface board 1646 is partially disposed within either the subject support or the longitudinal sledge 1616. The interface board 1646 would then move or translate along with the subject support or the longitudinal sledge 1616.

The interface board 1646 may be a PCB, a flat cable and/or the like, and may be mounted atop a supporting plate 1850. One or more of the interface board 1646 and the supporting plate 1850 may be configured to be received by the slot defined in the subject support apparatus 1600. For example, the supporting plate 1850 may be dimensioned such that it can be received by the slot, or may comprise grooves or ridges which allow it to translate smoothly along the slot.

The interface board 1646 is provided with a number of electrical connection points 1848 to the subject support. The electrical connection points may connect directly to the subject support, or may be connected via the longitudinal sledge 1616 (not shown).

The interface board 1646 may further comprise a connection point for connecting to the cable chain 1662, such that at least one electrical cable housed in the cable chain can electrically connect to the interface board 1646.

The cover 1614 may be configured to cover the electrical cables and/or the cable chain 1662, as shown in FIG. 18b.

In some implementations, the slot defined between the cover 1614 and the platform 1618 may be no wider than 8 mm, and may preferably be 6.6 mm as shown in FIG. 18c. This ensures that the slot itself does not pose a finger trapping risk to those utilising the apparatus. The interface board may be a 1.6 mm thick PCB board such that it may be received in the defined slot. The supporting plate may preferably be 2 mm thick, such that it may be received in the defined slot along with the interface board. Preferably, a gap of 1.5 mm may be left between the supporting plate 1850 and the platform 1618, and between the interface board 1852 and the cover 1614. In some embodiments, a flat cable may be used in addition to or as an alternative to a PCB board, in a similar manner.

For all of the implementations described above, in some embodiments, the operation of the actuators such as actuator 625 may be controlled by means of a software program executed by a processor. However, the operation of the actuator 625 may be manually controlled if desired.

According to embodiments of the present disclosure, the subject support of the subject support apparatus 114 may be tilted or rotated about the transverse (x-) axis 115 (pitch) and/or tilted or rotated about the longitudinal (y-) axis 113 (roll). Thus, according to the present disclosure, it is possible to position a patient 140 with an additional two degrees of freedom. The additional two degrees of freedom (pitch and roll) may be adjusted independently or in combination, and whilst the couch is in a neutral rotational position (yaw) or when it is in a rotated position. For patient's safety and comfort, the amount of pitch and roll is preferably limited to a predetermined maximum angle. Embodiments of the present disclosure provides an arrangement using simple mechanical elements to achieve one or more additional degrees of freedom in pitch and/or roll, the movement of which can be controlled straightforwardly due to the independent and predictable movement of the support surface in the transverse or longitudinal directions along the inclined surfaces of the guides. The simplicity of the arrangement allows the size of the couch to be kept relatively compact, enabling its centre of gravity to be kept low. The rotation of the support surface about the transverse and longitudinal axes can be arranged to pivot around the same point at the centre of the plate, and as such any compensations required in the x-, y- or z-direction may be minimised. The disclosed arrangement utilising opposing guides limits or prevents the movement of the plate 640, and therefore the subject support 212, to the direction of the axes of the first, and optionally second, guides. In other words, rotation around the z-axis is restricted, or locked, by the arrangement of the guides according to preferred embodiments. As such, additional stabilizing mechanism is not required to prevent accidental rotation around the z-axis while the support surface is rolled or pitched. The positions of the guides provide a wide base for receiving the weight of the support surface and the subject thereon, thereby improving the stability of the support surface. In some examples, the subject support or a section thereof may be pitched or rolled about an axis that is spaced apart from the isocenter 124 whilst a different section may be moved to compensate and maintain a portion of the couch 114 substantially at the isocenter 124. In this way, it is possible to maximise the spread of the radiation through the healthy tissue whilst maximising the dose of radiation that is delivered to the target region.

Embodiments of the present disclosure described above are only exemplary and many variations are possible, as will be apparent to the skilled person. Thus, the above description comprises examples comprising preferred and/or optional features of the disclosed embodiments, strict literal compliance with the meaning of the words is not intended and there may be other variations apparent to the skilled person that result in substantially the same or similar effects.

The following embodiments are also disclosed:
1. A subject support apparatus for a radiation therapy device, the apparatus comprising:
   a subject support, and
   a subject support receiving portion;
   wherein the subject support is configured to tilt relative to the subject support receiving portion, and
   a deformable guide comprising a first face and a second face, wherein, throughout a range of tilt configurations:
      the first face is arranged to be in contact, at least in part, with a longitudinal side wall of the subject support, and
      the second face is arranged to be in contact with a longitudinal exterior wall of the subject support receiving portion.
2. The apparatus of clause 1, wherein the deformable guide is elastically deformable.
3. The apparatus of clause 1 or 2, wherein the deformable guide comprises an expanded plastic.
4. The apparatus of any preceding clause, wherein the part of the first face arranged to be in contact with the longitudinal side wall of the subject support is less than 15 mm in length.
5. The apparatus of any preceding clause, wherein the deformable guide comprises a low friction coating.
6. The apparatus of any preceding clause, wherein the first face of the deformable guide comprises a recess.
7. The apparatus of any preceding clause, wherein the deformable guide comprises a channel extending longitudinally through the deformable guide.
8. The apparatus of any preceding clause, wherein the deformable guide is in a state of deformation where the subject support is in a neutral tilt position.
9. The apparatus of any preceding clause, wherein the deformation of the deformable guide increases as an angle between the subject support and the longitudinal exterior wall of the subject support receiving portion decreases.
10. The apparatus of any preceding clause, wherein the deformable guide expands outwardly from the apparatus as an angle between the subject support and the longitudinal exterior wall of the subject support receiving portion decreases.
11. The apparatus of any preceding clause, wherein:
   the deformable guide further comprises an upper face;
   the subject support comprises a subject support surface, said subject support surface protruding over the longitudinal wall of the subject support; and
   the upper face of the deformable guide being attached to an underside of the subject support surface.
12. The apparatus of any preceding clause, wherein the deformable guide is attached to the subject support and/or the subject support receiving portion.
13. The apparatus of any preceding clause, comprising a further deformable guide,
   said further deformable guide comprising a third face and a fourth face, wherein,
   throughout a range of tilt configurations:
      the third face is arranged to be in contact, at least in part, with a further longitudinal side wall of the subject support and
      the fourth face is arranged to be in contact with a further longitudinal exterior wall of the subject support receiving portion.
14. A radiotherapy device comprising the subject support apparatus of any of clauses 1 to 13.
15. A subject support apparatus for a radiation therapy device, the apparatus comprising:
   a subject support receiving portion comprising a longitudinal interior wall and a longitudinal exterior wall, the longitudinal exterior wall being inclined relative to a horizontal axis;
   a subject support, comprising:
      a longitudinal side wall, and
      a tabletop component comprising a surface and an extending edge, the extending edge extending beyond the longitudinal side wall,
   the subject support being tiltable about a tilt axis relative to the subject support receiving portion;
   wherein the longitudinal side wall is arranged to oppose the longitudinal interior wall of the subject support receiving portion; and
   wherein, at a maximally tilted position of the subject support, a distance between a lower surface of the extending edge and the longitudinal exterior wall increases with increasing displacement from a central longitudinal axis of the subject support.
16. The apparatus of clause 15, wherein the longitudinal exterior wall is inclined at a first angle relative to the horizontal axis and, at the maximally tilted position of the subject support, the extending edge is angled at a second angle relative to the horizontal axis, and wherein a difference between the first angle and the second angle is greater than a threshold angular difference.
17. The apparatus of clause 15 or clause 16, wherein the extending edge is in-plane with the surface of the tabletop component.
18. The apparatus of any of clauses 15 to 17, wherein the subject support receiving portion comprises a cover.
19. The apparatus of any of clauses 15 to 18 wherein the extending edge of the tabletop component further comprises a recess for receiving at least part of an accessory attached to the subject support.
20. The apparatus of clause 19, wherein the recess comprises an accessory surface located on a side of the subject support which is opposite to the surface of the tabletop component.
21. The apparatus of any of clauses 15 to 20, wherein the longitudinal side wall comprises a portion which does not oppose the longitudinal interior wall of the subject support receiving portion as the subject support is tilted about the tilt axis.
22. The apparatus of any of clauses 15 to 21, wherein the extending edge of the tabletop component of the subject support comprises a liquid directing feature for minimising liquid ingress into the subject support receiving portion.
23. The apparatus of clause 22, wherein the liquid directing feature comprises a groove configured to interrupt flow of liquid from the subject support towards the subject support receiving portion.
24. The apparatus of clause 23, wherein the groove is located on the underside of the extending edge, and arranged to provide capillary action.
25. The apparatus of clause 22 or 23, wherein the liquid directing feature runs substantially parallel to the tilt axis.
26. The apparatus of any of clauses 22 to 25, wherein the liquid directing feature is configured to direct liquid away from a gap where the subject support is received by the subject support receiving portion, the gap being defined by the longitudinal side wall and the longitudinal interior wall.
27. The apparatus of clause 26, wherein the gap is at a location where the subject support emerges from or retracts within the subject support receiving portion as the subject support tilts about the tilt axis.
28. A radiotherapy device comprising the subject support apparatus of any of clauses 15 to 27.
29. A subject support apparatus for a radiation therapy device, the apparatus comprising:
   a subject support comprising a first longitudinal side wall and a second longitudinal side wall, the first longitudinal side wall and a second longitudinal side wall each having a radius of curvature defined with respect to a tilt axis, the subject support having a maximum width less than or equal to double the radius of curvature; and
   a subject support receiving portion comprising a first longitudinal interior wall and a second longitudinal interior wall, each having a radius of curvature defined with respect to the tilt axis, the first longitudinal interior wall and the second longitudinal interior wall being arranged to oppose the first longitudinal side wall and the second longitudinal side wall of the subject support, respectively;
   wherein the subject support is configured to tilt about the tilt axis relative to the subject support receiving portion; and
   wherein the radius of curvature of the first and second longitudinal interior walls is greater than the radius of curvature of the first and second longitudinal side walls.
30. The apparatus of clause 29, wherein the radius of curvature of the first and second longitudinal interior walls is less than 8 mm greater than the radius of curvature of the first and second longitudinal side walls.
31. The apparatus of clause 29 or clause 30, wherein the subject support receiving portion is configured such that a tilting of the subject support about the tilt axis causes the first longitudinal side wall to emerge from the subject support receiving portion, and causes the second longitudinal side wall to retract into the subject support receiving portion or, alternatively, causes the second longitudinal side wall to emerge from the subject support receiving portion, and causes the first longitudinal side wall to retract into the subject support receiving portion.
32. The apparatus of any of clauses 29 to 31, wherein a portion of at least one of the first longitudinal wall and the second longitudinal side wall protrudes from the subject support receiving portion in all tilt configurations of the subject support.
33. The apparatus of any of clauses 29 to 32, wherein the portion of the at least one of the first longitudinal side wall and the second longitudinal side wall of the subject support comprises an additional recess for receiving at least part of an accessory attached to the subject support.
34. The apparatus of any of clauses 29 to 33, wherein the subject support comprises a liquid directing feature for channeling fluid away from the subject support receiving portion.
35. The apparatus of clause 34, wherein the liquid directing feature is configured to interrupt a flow of liquid from the subject support towards a gap, the gap being defined by either the first longitudinal side wall and the first longitudinal interior wall, or by the second longitudinal side wall and the second longitudinal interior wall.
36. The apparatus of clause 35, wherein the liquid directing feature is configured to direct the flow of liquid away from the gap.
37. The apparatus of any of clauses 34 to 36 when dependent on clause 32, wherein the liquid directing feature comprises a lip which protrudes from a surface of the recess.
38. The apparatus of any of clauses 34 to 36, wherein the liquid directing feature comprises a channel located within the subject support receiving portion.
39. The apparatus of clause 38, wherein the channel is configured to direct liquid towards a longitudinal end of the apparatus.
40. The apparatus of any of clauses 34 to 36, wherein the liquid directing feature comprises a groove configured to exhibit capillary action.
41. The apparatus of any of clauses 29 to 40, wherein the subject support further comprises:
   a top surface between the first longitudinal side wall and the second longitudinal side wall, the top surface being parallel to the ground when the subject support is in a neutral tilting position; and
   a base surface between the first longitudinal side wall and the second longitudinal side wall, the base surface being on an opposite side of the subject support as the top surface.
42. The apparatus of clause 41, wherein the base surface is curved with the radius of curvature of the first and second longitudinal side walls.
43. The apparatus of clause 41, wherein the base surface is substantially flat.
44. A radiotherapy device comprising the apparatus of any one of clauses 29 to 43.
45. A subject support apparatus for a radiation therapy device, the apparatus comprising:
   a platform;
   a subject support movable relative to the platform and supported by the platform; and
   an interface board movable relative to the platform,
   wherein the interface board is physically coupled to the subject support such that a translation of the subject support relative to the platform causes a translation of the interface board; and
   wherein the interface board is configured to electrically connect the subject support to an electrical cable.
46. The apparatus of clause 45, wherein the platform is arranged to support the interface board.
47. The apparatus of clause 45 or 46, further comprising a slot configured to receive the interface board.
48. The apparatus of clause 47, wherein the slot is defined by a gap between the platform and a cover, the cover being disposed generally above the platform.
49. The apparatus of clause 48, wherein the cover is configured to cover an electrical cable.
50. The apparatus of any of clauses 47 to 49, wherein a movement of the subject support relative to the platform at least partially exposes said slot.
51. The apparatus of any of clauses 47 to 50, wherein the slot is configured to accommodate the movement of the interface board relative to the platform.
52. The apparatus of any of clauses 47 to 51, wherein the interface board is configured to translate along said slot.
53. The apparatus of any of clauses 47 to 52, wherein the slot is less than or equal to 8 mm in width.
54. The apparatus of any of clauses 45 to 53, wherein the interface board is mounted to the subject support.
55. The apparatus of any of clauses 45 to 54, wherein the interface board comprises a PCB and/or a flat cable.
56. The apparatus of clause 55, wherein the interface board further comprises a supporting plate for supporting the PCB and/or the flat cable.
57. The apparatus of any of clauses 45 to 56 wherein the apparatus further comprises a cable chain configured to contain an electrical cable.
58. The apparatus of clause 57, wherein the cable chain is configured to maintain a plurality of cables.
59. The apparatus of clause 57 or clause 58, wherein the cable chain is disposed between a cover and the platform.
60. The apparatus of any of clauses 45 to 59, wherein the platform is configured to route one or more electrical cables within the platform.
61. The apparatus of any of clauses 45 to 60, wherein the interface board is configured to electrically couple to a tilting module configured to control a tilt of the subject support relative to the platform.
62. The apparatus of any of clauses 45 to 61, wherein the interface board is configured to electrically connect the subject support to a plurality of cables.
63. The apparatus of any of clauses 45 to 62 wherein the interface board comprises a plurality of electrical connections to the subject support.
64. A radiotherapy device comprising the apparatus of any one of clauses 45 to 63.
65. A device for a subject support apparatus, the device comprising:
   a linkage for moving a platform, the linkage being movable between an extended pose and a retracted pose; and
   a base movably coupled to the linkage, wherein the base comprises a linkage receiving region configured to receive at least a portion of the linkage when the linkage is in the retracted pose; and
   wherein said portion of the linkage extends beyond the base when the linkage is in the extended pose.
66. The device of clause 65, wherein the linkage receiving region comprises a recess.
67. The device of clause 65 or 66, wherein the linkage receiving region comprises a cut-out portion of the base.
68. The device of any of clauses 65 to 67, wherein the linkage receiving region is defined by having a thickness less than a total thickness of the base, measured vertically.
69. The device of any of clauses 65 to 68, wherein the base is movably coupled to the linkage via a coupling disposed in the linkage receiving region.
70. The device of any of clauses 65 to 69, wherein the linkage comprises a first member movably coupled to the base, and wherein the base is configured to receive at least a portion of the first member.
71. The device of clause 70, wherein the first member has a length and a width, and wherein the received portion of the first member extends along the length of the first member.
72. The device of clause 70 or 71, wherein the linkage further comprises a second member movably coupled to the first member, wherein the second member is movably couplable to a platform.
73. The device of any of clauses 65 to 72, wherein the linkage is configured to adjust at least one of height, pitch, roll and yaw of a subject support platform.
74. The device of clause 73, wherein the linkage is disposed under a platform of the subject support apparatus.
75. The device of clause 73 or 74, wherein the linkage and the base are configured in a stacked arrangement with a platform of the subject support apparatus.
76. The device of any of clauses 65 to 75, wherein the base is configured to be movably couplable to a floor of a treatment room.
77. The device of any of clauses 65 to 76, wherein the linkage is configured to raise and/or lower a platform.
78. The device of any of clauses 65 to 77, wherein the recess extends towards a bottom surface of the base.
79. A subject support apparatus comprising the device of any one of clauses 65 to 78.
80. The subject support apparatus of clause 79, further comprising a platform.

1. A subject support apparatus for a radiation therapy device, the subject support apparatus comprising:
a subject support;
a subject support receiving portion, wherein the subject support is configured to tilt relative to the subject support receiving portion; and
a deformable guide comprising a first face and a second face, wherein, throughout a range of tilt configurations:
a portion of the first face is arranged to be in contact, at least in part, with a longitudinal side wall of the subject support; and
the second face is arranged to be in contact with a longitudinal exterior wall of the subject support receiving portion.

2. The subject support apparatus of claim 1, wherein the deformable guide is elastically deformable.

3. The subject support apparatus of claim 1, wherein the deformable guide comprises an expanded plastic.

4. The subject support apparatus of claim 1, wherein the portion of the first face arranged to be in contact with the longitudinal side wall of the subject support is less than 15 mm in length.

5. The subject support apparatus of claim 1, wherein the deformable guide comprises a low friction coating.

6. The subject support apparatus of claim 1, wherein the first face of the deformable guide comprises a recess.

7. The subject support apparatus of claim 1, wherein the deformable guide comprises a channel extending longitudinally through the deformable guide.

8. The subject support apparatus of claim 1, wherein the deformable guide is in a state of deformation when the subject support is in a neutral tilt position.

9. The subject support apparatus of claim 8, wherein the deformation of the deformable guide increases as an angle between the subject support and the longitudinal exterior wall of the subject support receiving portion decreases.

10. The subject support apparatus of claim 1, wherein the deformable guide expands outwardly from the subject support apparatus as an angle between the subject support and the longitudinal exterior wall of the subject support receiving portion decreases.

11. The subject support apparatus of claim 1, wherein:
the deformable guide further comprises an upper face;
the subject support comprises a subject support surface, the subject support surface protruding over the longitudinal side wall of the subject support; and
the upper face of the deformable guide being attached to an underside of the subject support surface.

12. The subject support apparatus of claim 1, wherein the deformable guide is attached to at least one of the subject support or the subject support receiving portion.

13. The subject support apparatus of claim 1, comprising a further deformable guide, wherein the further deformable guide comprises a third face and a fourth face, and wherein, throughout a range of tilt configurations:
a portion of the third face is arranged to be in contact, at least in part, with a further longitudinal side wall of the subject support; and
the fourth face is arranged to be in contact with a further longitudinal exterior wall of the subject support receiving portion.

14.-17. (canceled)

18. A radiation therapy device comprising a subject support apparatus, the subject support apparatus comprising:
a subject support;
a subject support receiving portion, wherein the subject support is configured to tilt relative to the subject support receiving portion; and
a deformable guide comprising a first face and a second face, wherein, throughout a range of tilt configurations:
a portion of the first face is arranged to be in contact, at least in part, with a longitudinal side wall of the subject support; and
the second face is arranged to be in contact with a longitudinal exterior wall of the subject support receiving portion.

19. The radiation therapy device of claim 18, wherein the deformable guide is elastically deformable.

20. The radiation therapy device of claim 18, wherein the first face of the deformable guide comprises a recess.

21. The radiation therapy device of claim 18, wherein the deformable guide comprises a channel extending longitudinally through the deformable guide.

22. The radiation therapy device of claim 18, comprising a further deformable guide,
wherein the further deformable guide comprises a third face and a fourth face, and wherein, throughout a range of tilt configurations:
a portion of the third face is arranged to be in contact, at least in part, with a further longitudinal side wall of the subject support; and
the fourth face is arranged to be in contact with a further longitudinal exterior wall of the subject support receiving portion.

23. The radiation therapy device of claim 18, wherein the deformable guide expands outwardly from the subject support apparatus as an angle between the subject support and the longitudinal exterior wall of the subject support receiving portion decreases.

24. The radiation therapy device of claim 18, wherein the deformable guide is configured to:
be in a state of partial deformation when the subject support is in a neutral tilt position;
increase in deformation as an angle between the subject support and the longitudinal exterior wall of the subject support receiving portion decreases; and
expand outwardly from the subject support apparatus as the deformation increases, thereby pushing any object in contact with the deformable guide away from a gap between the subject support and the subject support receiving portion.
